(19) United States
(12) Patent Application Publication
(10) Pub. No.: US 2026/0207052 A1
(43) Pub. Date: Jul. 23, 2026

(54) SYSTEMS AND METHODS FOR REMOTE OPTICAL MONITORING OF INTRAOCULAR PRESSURE

(71) Applicant: Smartlens, Inc., Mountain View, CA (US)

(72) Inventors: Ahmet Taylan YAZICI, Mountain View, CA (US); Savas KOMBAN, Mountain View, CA (US); Sevda AGAOGLU, Mountain View, CA (US); Murat BADAY, Mountain View, CA (US)

(73) Assignee: Smartlens, Inc., Mountain View, CA (US)

(21) Appl. No.: 19/318,242

(22) Filed: Sep. 3, 2025

Related U.S. Application Data
(63) Continuation of application No. PCT/US24/19264, filed on Mar. 8, 2024.
(60) Provisional application No. 63/489,680, filed on Mar. 10, 2023.

(51) Int. Cl. A61B 3/16; A61B 3/00; A61B 3/14

(52) U.S. Cl. CPC A61B 3/16; A61B 3/005; A61B 3/14

(57) ABSTRACT

Intraocular pressure (IOP) of an eye is determined by retrieving a first image data, analyzing the first image data using a trained deep neural network, and annotating the first image data with the analysis produced by the trained deep neural network.

### CROSS-REFERENCE

This application is a continuation of PCT Application No. PCT/US24/19264, filed Mar. 8, 2024, which claims benefit of U.S. Provisional Patent Application No. 63/489,680, filed Mar. 10, 2023, which is entirely incorporated herein by reference.

The present application is related to U.S. Provisional No. 62/790,752, filed Jan. 10, 2019, entitled “METHOD AND DEVICE FOR REMOTE OPTICAL MONITORING OF INTRAOCULAR PRESSURE” and is also related to U.S. patent application Ser. No. 16/124,630, filed Sep. 7, 2018, entitled “CLOSED MICROFLUIDIC NETWORK FOR STRAIN SENSING EMBEDDED IN A CONTACT LENS TO MONITOR INTRAOCULAR PRESSURE,” the contents of each of which are incorporated herein in their entirety by reference.

### FIELD OF THE INVENTION

The present disclosure is related to a system and methods of using a wearable optical imaging sensor system for measuring intraocular pressure.

### BACKGROUND

Glaucoma is the second most common cause of blindness in the global world. It is a multifactorial disease with several risk factors, of which intraocular pressure (IOP) is the most important. IOP measurements are used for glaucoma diagnosis and patient monitoring. IOP has wide diurnal fluctuation, and is dependent on body posture, so the occasional measurements done by the eye care expert in a clinic can be misleading.

### SUMMARY

Provided herein are devices, systems, and methods for determining an intraocular pressure (IOP) of an eye. Aspects of the disclosure describe a method of determining an IOP of an eye, comprising: obtaining or receiving a first image of at least two markers embedded in the eye detected with a first detector and a second image of the at least two markers detected with a second detector, where an optical axis of the first detector is at an angle with respect to an optical axis of the second detector; and determining the IOP of the eye from a position of one or more of the at least two markers from the first image or the at least two markers from the second image.

In some embodiments, the at least two markers are embedded on a surface of the eye, in the cornea of the eye, in the lens of the eye, in a tissue adjacent to the eye, or any combination thereof. In some embodiments, the at least two markers comprise fluorescent markers. In some embodiments, the method further comprises providing a light source to the at least two markers embedded in the eye and obtaining or detecting the first image or the second image. In some embodiments, the light source comprises a light emitting diode. In some embodiments, the light source, first detector, second detector, or a combination thereof, are contained at least partially within a chassis. In some embodiments, the chassis comprises eyewear, eyeglasses, goggles, heads up display, virtual reality eyewear, augmented reality eyewear, or any combination thereof.

In some embodiments, the first image of the at least two markers embedded in the eye is obtained or detected by the first detector through a first polarizing filter, and wherein the second image of the at least two markers embedded in the eye is obtained or detected by the second detector through a second polarizing filter.

In some embodiments, the at least two markers comprise a particle. In some embodiments, the at least two markers comprise a first pair of markers and a second pair of markers. In some embodiments, the IOP of the eye is determined from a ratio of a distance between a first marker and a second marker of the first pair of markers and a distance between a third marker and a fourth marker of the second pair of markers. In some embodiments, the position of the at least two markers is provided as an input to a machine learning algorithm or predictive model, where the machine learning algorithm or predictive model is trained to provide an output related to the IOP of the eye.

Described herein is a system for determining an IOP of an eye, comprising: a detector optically coupled to an eye, where the detector is configured to detect a signal of at least two markers embedded in the eye; and one or more processors electrically coupled to the detector configured to process the signal of the two or more markers embedded in the eye and determine the TOP of the eye from a position of the two or more markers. In some embodiments, the at least two markers are positioned at a distance of up to about 6 millimeters from each other.

In some embodiments, the system further comprises a light source optically coupled to the eye of the subject. In some embodiments, the light source comprises a light emitting diode (LED). In some embodiments, the light source and detector are contained at least partially in a chassis, where the chassis is positioned at a distance up to about 2 centimeters from a tangential surface of the eye. In some embodiments, the chassis comprises eyewear, eyeglasses, googles, heads up display, virtual reality eyewear, augmented reality eyewear, or any combination thereof.

In some embodiments, the detector comprises a first detector and a second detector. In some embodiments, an optical axis of the first detector is at an angle with respect to an optical axis of the second detector. In some embodiments, the detector comprises a camera, a charge coupled device (CCD) sensor, complementary metal oxide semiconductor (CMOS) sensor, or any combination thereof.

In some embodiments, the system comprises any of the devices used in the methods described herein.

Provided herein is a system for determining an IOP of an eye, comprising: one or more processors and a memory storing one or more programs for execution by the one or more processors, the one or more programs comprising instructions to: (i) obtain or receive one or more images of at least two markers embedded in the eye, where the one or more images are detected with a first detector and a second detector, and where an optical axis of the first detector is at an angle with respect to an optical axis of the second detector; and (ii) determine the TOP of the eye from a distance between the at least two markers in the one or more images. In some embodiments, the at least two markers are positioned at a distance of up to about 6 millimeters from each other. In some embodiments, the one or more images are obtained or received from a server, cloud base storage, eyewear device, or any combination thereof. In some embodiments, the eyewear device comprises virtual reality eyewear, augmented reality eyewear, or a combination thereof. In some embodiments, the one or more processors are on the eyewear device, a remote processing device, a remote server, a cloud server, or any combination thereof. In some embodiments, the system comprises any of the devices used in the methods described herein.

Described herein is a method of training a machine learning algorithm or predictive model to determine an IOP of an eye, comprising: receiving or obtaining one or more images of at least two markers embedded in one or more eyes and a corresponding IOP of the one or more eyes; and training an untrained or partially untrained machine learning algorithm or predictive model with the one or more images of the at least two markers embedded in the one or more eyes and the corresponding IOP of the one or more eyes thereby producing a trained machine learning algorithm or trained predictive model. In some embodiments, the untrained or partially untrained machine learning algorithm or predictive model is further trained on a distance between the at least two markers in the one or more images of the at least two markers embedded in the one or more eyes.

In some embodiments, the one or more images of the at least two markers embedded in the one or more eyes is detected with a first detector and a second detector, and where an optical axis of the first detector is at an angle with respect to an optical axis of the second detector. In some embodiments, the at least two markers are positioned at a distance of up to about 6 millimeters from each other. In some embodiments, the at least two markers are on a surface of the eye, in the cornea of the eye, in the lens of the eye, in a tissue adjacent to the eye, or any combination thereof.

In some embodiments, the systems, described elsewhere herein, are used to train the machine learning algorithm or predictive model to determine an IOP of an eye.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a diagram showing variation of a side view of corneal thickness in accordance with an example embodiment described herein.

FIG. 2 shows a table of structural eye parameters of a model eye in accordance with an example embodiment described herein.

FIG. 3 illustrates side views of tools used in the devices, systems, and methods of the subject matter in accordance with an example embodiment described herein.

FIG. 4A illustrates a schematic cross section of a cornea with two implants positioned on different sides of the central axis of the cornea in accordance with an example embodiment described herein.

FIG. 4B illustrates IOP as a function of a calculated distance between corneal implants in accordance with an example embodiment described herein.

FIG. 5 illustrates a calculated distance between corneal implants as a function of positioning angle in degrees in accordance with an example embodiment described herein.

FIG. 6 illustrates a frontal image of an eye, with the positions of fluorescent implants on the cornea and sclera in accordance with an example embodiment described herein.

FIG. 7 illustrates an imaging goggle, where two cameras (front and side looking) may be used to image the cornea and implants, in accordance with an example embodiment described herein.

FIG. 8 illustrates a schematic view of the side of an eye, with the positions of two fluorescent implants on the cornea in accordance with an example embodiment described herein.

FIG. 9 illustrates a schematic view of the front of an eye, with the positions of four fluorescent implants on the cornea in accordance with an example embodiment described herein.

FIG. 10 illustrates an example of measured distances of two fluorescent particles on a model cornea when subjected to various internal pressures, in accordance with an example embodiment described herein.

FIG. 11 illustrates the positioning of a pair of observation cameras relative to imaging illumination, corneal implants, and the eye in accordance with an example embodiment described herein.

FIG. 12 illustrates an example flow diagram of training a deep neural network in accordance with an example embodiment described herein.

FIG. 13 illustrates an example flow diagram of using the deep neural network in accordance with an example embodiment described herein.

### DETAILED DESCRIPTION

Subjects with sever glaucoma require continual monitoring of their intraocular pressure (IOP) to maintain the health of their optic nerve to prevent degradation and loss of eyesight. Conventional methods of measuring IOP require subjects to travel to their ophthalmologist and/or optometrist to measure their IOP using an in-office device which can be cumbersome and inconvenient to subjects that require frequent or continual monitoring of IOP. Thus, there exists an unmet need of devices, systems, and/or methods that can conveniently and accurately measure IOP.

Increases in IOP of an eye result in bulging of the cornea and consequently changes in the radius of curvature of the cornea and/or eye. The IOP changes affect corneal topography, causing changes in corneal radius and apex height with respect to the corneal periphery. By measuring a difference in corneal radius at a resolution and/or scale of about 4 micrometers, a change of about 1 mmHg IOP can be determined and/or monitored. Thus, there exist an unmet need for an IOP measuring device, system, and/or method that can take multiple measurements and/or continuously monitor the curvature of a subject's eye e.g., throughout the day as the subject goes through their normal routine to determine changes IOP that would otherwise require a visit to an ophthalmologist and/or optometrists' office. There is also a need for a device that has sufficient sensitivity and/or accuracy in measuring curvature of the eye to produce reliable data for accurate determinations of IOP. There is still further a need for such a device to operate in a manner that does not interfere with a patient's normal vision and activities. There is still further a need for a device that can operate reliably while a patient carries on their normal daily activities, and the device does not require a particular critical position or alignment relative to the patient's eyes. There is still a further need that the device can be user friendly.

Devices, systems, and methods are described herein that may measure, obtain, and/or determine IOP of an eye with eyewear placed at a distance from the eye. In some cases, the eyewear may comprise one or more illuminators, one or more image sensors, or a combination thereof. The combination of the one or more illuminator(s) and one or more image sensor(s) may eliminate one or more ambient lighting changes and/or misalignment errors, that would otherwise produce noise and error in a measurement of corneal radius and/or resulting IOP determination(s). The devices, systems, and/or methods described elsewhere herein, may measure a change of a radius of curvature of a cornea (e.g., at about 4 micrometers per about 1 mmHg change in IOP for an adult cornea). This change along the surface normal of the cornea can be difficult to measure with a conventional visible light imaging system. One or more particles and/or markers (e.g., fluorescent or dye doped microparticles) can be implanted in predetermined positions on the cornea to convert the change of curvature of the cornea into a vertical and/or horizonal displacement of the one or more particles and/or markers. The change in the corneal shape upon a change in IOP can result in a change position and/or the distances between corneal particle and/or marker implants that may be measured by imaging the particle and/or marker positions. In some cases, the vertical and/or horizontal displacement of the one or more particles and/or one or more markers may be measured, detected, and/or determined by acquiring one or more images of the one or more implanted particles with the one or more image sensors. The one or more images of the one or more implanted particles may be processed with image processing methods, one or more machine learning algorithm, one or more predictive models, or any combination thereof, described elsewhere herein, to measure a position (e.g., a relative position and/or an inter-particle distances) between the one or more particles implanted in the cornea. The optical design may allow image processing and sensor fusion. In some cases, the markers can be a type of sensor. The markers can be a type of sensor that signals its own position. The position can be determined by combining image processing and sensor data. The marker can also be a magnetic particle or of similar nature. The measured changes in position and/or inter particle distance may be used in a calculation using a machine learning program, a learning neural network, an artificial intelligence program, other analytic computational program, or any combination thereof, to relate the measured changes in position of the one or more particles to a change in corneal radius. In some instances, the change in the radius of the cornea may be converted to a change in IOP of an eye. The methods, described elsewhere herein, may use a preliminary characterization of the corneal thickness and corneal topography where the radius of curvature at a known IOP is acquired by conventional ophthalmologic methods (e.g., Goldmann Applanation Tonometry (GAT), tonopen tonometry, pneumotonometry, non-contact or air puff tonometry, Dynamic Contour Tonometry (DCT), or any combination thereof). Corneal curvature data of a subject obtained with eyewear, described elsewhere herein, may then be compared to the dataset of known IOP and corresponding corneal curvature(s) to calculate the IOP. In some cases, the dataset of known IOP and corresponding corneal curvature may be used to train a one or more partially untrained and/or untrained machine learning algorithms and/or predictive models. The dataset may be processed and/or manipulated by a computational device such as a cell phone, personal computer, laptop computer, server, cloud processing server, or any combination thereof. The present disclosure, in some embodiments, describes a wearable optical device that measures IOP through image acquisition from one or more image sensors and uses the image data along with a reference data for a particular individual to accurately determine the IOP.

These and other objectives may be met using the methods, devices, and/or systems described herein. In various embodiments, the present disclosure describes a method for measuring IOP remotely. The method can include implantation of fluorescent beads at specific locations in the cornea, and one or more of an apparatus for imaging of the positions of the fluorescent particles using a fluorescence imaging system which comprises an excitation light source, an emission filter, a camera, or any combination thereof. A system can include the apparatus for imaging the relative positions of the fluorescent particles implanted into the cornea and a microcontroller configured to receive and/or obtain the measured positions of particles for measuring the intraocular pressure (IOP) of an eye. In various embodiments, the IOP may be determined using position measurements of fluorescent dye doped microparticles. In some embodiments, one or more of these elements may be replaced with an equivalent element. In some embodiments, the fluorescent dye doped microparticles may be replaced with colored microbeads. The device for the readout may be a pair of goggles, glasses, or other eye wear.

In some embodiments, there may be an eyewear device for measuring intraocular pressure (IOP). The device may have a frame, a first lens mounted to the frame such that the lens may be in the field of view of a person wearing the eyewear device. The eyewear device may have a first illumination source positioned to illuminate the eye of a user with an excitation wavelength, typically green, an emission filter in front of the camera to eliminate the transmission of excitation light, a first image sensor positioned to capture images of the eye of the user, a first communication portal being in electronic or signal communication with a computational device, or any combination thereof.

In some embodiments, there may be a method of training an image processing pipeline. The method can involve collecting personalized ophthalmologic data on a user's anatomy and a user's corneal properties at a known IOP, collecting personalized data from an eyewear device for measuring IOP, and using at least one computational mode and ray tracing under one or more geometric configurations to generate at least one set of training data for a neural network components pipeline. In various embodiments, the computational device may be a cell phone, a tablet, a laptop computer, or any combination thereof. The computational device may be attached to the wearable eyewear device.

In some embodiments, the disclosure describes a method of determining the intraocular pressure (IOP) of an eye. The method may involve retrieving a first image data, analyzing the first image data using a trained deep neural network, and annotating the first image data with the analysis produced by the trained deep neural network.

The present disclosure describes wearable eyewear, systems, and methods for measuring the cornea of an eye and determining the intraocular pressure of the measured eye based on the changes in the curvature of the cornea. The disclosure describes corneal implants, eyewear, and/or computational devices that may calculate IOP values based on cornea deformation data collected by the eyewear. The disclosure also describes methods that may calculate the IOP. Descriptions herein which may use the terms eyewear device or eyewear are meant to be used interchangeably, and reference to either an eyewear device or eyewear is understood to mean any of the wearable eye wear systems, apparatus, and devices, as described herein, unless context specifically indicates otherwise.

The eyewear as described herein may take a variety of forms. The form factor may be one of choice for a user, or one for the user's optometrist or other professional medical person responsible for the user's eye health. In some embodiments, the form factor may include a frame and a lens. The frame may be one where the user may wear in front of his eyes (note the use of male or female pronouns may be distributed herein randomly and is interchangeable for a human subject and/or patient). The disclosed technology is not dependent on the gender of the user. The interchanging use of the gender of the user or other persons described herein is simply for the convenience of the applicant. The frame may be any sort of eyewear frame used for modern eyewear, including frames for sunglasses, vision correction glasses, safety glasses, goggles of all types (e.g., Swimming, athletic, safety, skiing, and so on). The frame may be suitable for a single lens for one eye, a lens for two eyes (e.g., a visor), or a single lens and an eye cover (such as for persons with amblyopia or who may suffer from the loss of one eye). The lens may be a prescription lens for vision correction, a clear or tinted lens for appearance, or an opaque lens that covers the eye. In some embodiments, the lens may have a defined area for the field of view of the user. The field of few may be clear to avoid blocking the vision of the user. The various elements of the eyewear device may be place on the periphery of the lens, on the frame, or a combination thereof. The frame or lens may have flanges or other protrusions and/or tabs for the attachment of image sensors, light sources, battery, computational devices, any other component suitable for the use with the present disclosure, or any combination thereof.

The wearable eyewear may have one or more image sensors positioned to face the eye(s) of the user such that the image sensor may capture an image of the eye. The image sensor may be a camera, a CCD (charge coupled device), CMOS (complementary metal oxide semiconductor), or other image capture technology. The wearable eyewear may have one or more light sources for projecting light at the eye. In some embodiments, the light source may be a form of illumination that produces specific wavelengths of light. The light emission may be at a shallow angle to the curvature of the cornea, and projected outside the lens portion of the eye such that the light does not interfere with the user's normal vision. In some embodiments the light source may be a LED (light emitting diode), and in other embodiments the light source may be any light generating technology now known or still to be developed.

In various embodiments, the light source(s) and image sensor(s) may be positioned so that images captured by the image sensor are able to ignore ambient light, glare, other optical artifacts, or any combination thereof, that might interfere with the accurate reading of the change in cornea curvature. The light source and the image sensor may use one or more polarizing filters to substantially reduce and/or eliminate light of a particular polarization, wavelength, intensity, or any combination thereof, such that the captured image may have greater reliability and less signal noise. In some cases, the eyewear may have a light sensor to help regulate when the ambient lighting conditions are appropriate for taking a suitable image of the eye to determine cornea curvature. The images captured by the image sensors may be stored locally for a period of time and/or transmitted to a computational device via a communication portal, described elsewhere herein.

In some embodiments, the communication portal may be an antenna for wireless transmission of data to a computational device. The communication portal may send and receive information, such as sending image data, and/or receiving dosing information for a drug delivery device. In various embodiments, the computational device may be a cell phone, a tablet computer, a laptop computer, a personal computer, any other computational device, or any combination thereof, a user may select to carry out program (App) functions for the eyewear device. In some embodiments, the computational device may be resident on the eyewear. In some embodiments, the communication portal may be a wired connection between the image sensors, the light sources, the computational device, a power supply for all the electrical components, or any combination thereof. In some cases, the communication portal may connect the eyewear to the cloud.

In some embodiments, the disclosure describes a method for determining the IOP of an eye. In some embodiments, the method may use a basic operation pipeline. The pipeline may receive image data from a variety of sources. In some embodiments the image data may come from the eyewear as it is worn by a user and/or subject. In some embodiments the image data may come from a database having stored ophthalmologic data of the user and/or subject at a fixed point in time. In some embodiments the images may be anatomic data of a user from a fixed point in time. In some embodiments, some or all the available image data may be used in a deep neural network with an image processing front-end. The image processing front-end may derive or calculate an IOP reading. In some embodiments, the IOP reading may be updated at video data rates, providing a quasi-real time output.

In some embodiments, the data pipeline may cause an image sensor to change exposure levels, gain, brightness, contrast, or any combination thereof, in order to capture non-saturated images. The images may be passed through a threshold filter to reduce or eliminate background noise. Some high-resolution images may be stored in a temporary memory for rapid processing, while blurry and/or low-resolution images are formed. The low-resolution images may then be passed through a match filter and/or feature detection filter to pinpoint spots corresponding to one or more particles and/or markers illuminated by the illumination/light sources in the various captured images. The coarse locations of the one or more particles and/or markers may then be used to segment the high-resolution images and perform peak fitting algorithms to individually determine the positions and widths of each peak in the images. The results of the peak locations and widths may then be used with the previously trained neural network, which may then be used to estimate cornea coordinate and radius of curvature. A nonlinear equation solver may be used to convert the radius of curvature into an IOP reading.

As described herein, a wearable eyewear device may be coupled to a computational device to measure the IOP of a user's eye. The user and/or subject may be a person wearing the eyewear unless the context of the usage clearly indicates otherwise.

Reference is made herein to various components and images. The use of the references is to help guide the reader in a further understanding of the present disclosure. In particular, while the singular version of a noun is often used, it should be understood that the embodiments fully consider plural numbers of components and images to also be within the scope of the disclosure.

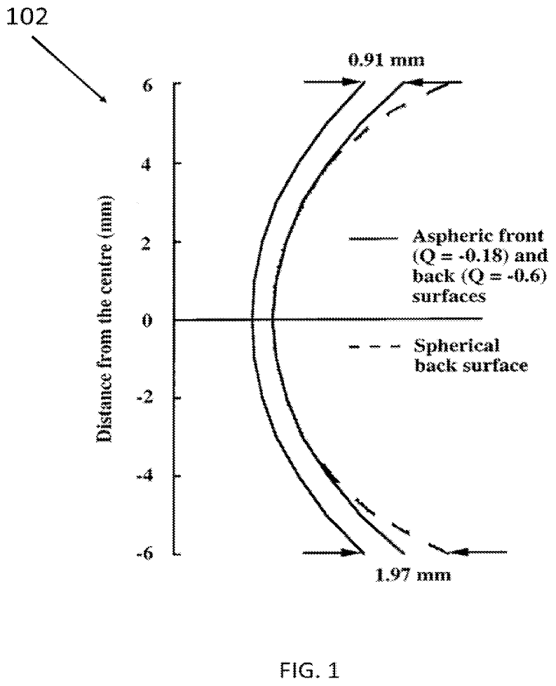

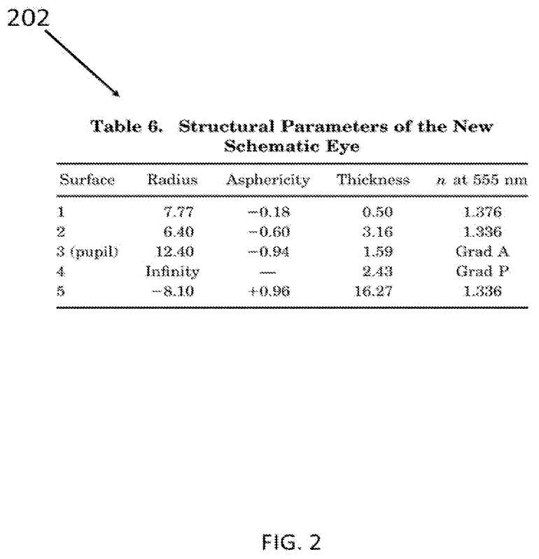

Referring now to the FIG. 1, a cross-sectional view of the human cornea model 102 is shown. The aspheric model parameters are given in FIG. 2. The diagram of FIG. 1 shows the distance in millimeters (mm) on the y-axis, and the delta in corneal curvature or surface deflection on the x-axis. The cornea may be defined by aspheric surfaces whose parameters are given in FIG. 2 and deviates from a spherical surface. The corneal thickness may be about 0.91 mm at the edges and decreases to about 0.6 mm at the apex. Upon a change in the IOP, the cornea shape can be treated as fixed at the edges where it meets the sclera leading to a change in the radius of curvature of the cornea. The front surface is illustrated with the solid line, while the back surface is illustrated with a dashed line.

FIG. 2 shows a table 202 of the structural parameters of the new schematic eye. The model eye shows the directions of angle alpha and pupil decentration. The table gives the radii of curvature for the front and back surfaces of the cornea as well as the aspheric parameters and indices of refraction.

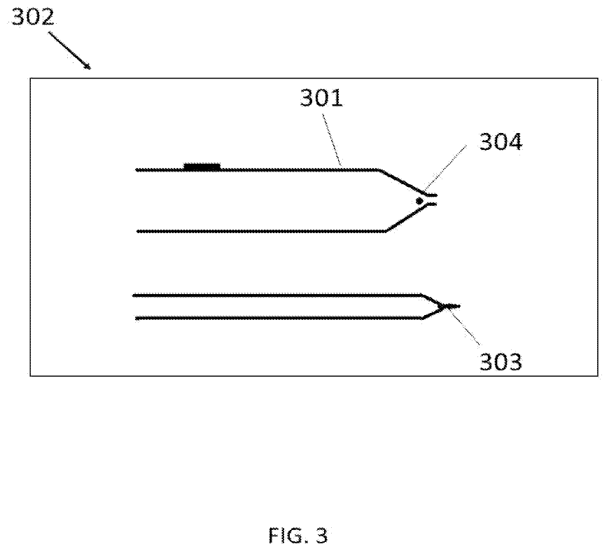

Now referring to FIG. 3, a variety of tools 302 are illustrated for the creation of a pocket to contain the particle, marker, and/or fluorescent bead (304), described elsewhere herein. In some embodiments, an implantation area may be selected on the surface of the eye. The location of the implantation area may be on the cornea, the lens, other surface areas of the eye, or any combination thereof. In some embodiments, one or more implantation areas may be in tissue adjacent to the eye to serve as a fiducial for measurements. In some embodiments, the implantation area in the cornea to house one or more particles, markers, and/or fluorescent bead(s) may be created with a femtosecond laser. The laser may be used to penetrate the cornea to a desired corneal depth, so that the pocket created may be sufficiently large to hold a fluorescent bead, particle, and/or marker. In some embodiments, a tunnel may also be made with a laser in accordance with the guide dimensions, which may enter at the closest horizontal distance to the pocket created at the implantation area. The incision created with the laser in the eye may be opened with a spatula or other instrument, and the fluorescent bead(s), particle, and/or marker, may be placed into the pocket or tunnel with the help of a guide.

The corneal incision to be created as a bead, particle, and/or marker pocket may also be created with a microsurgery knife 303 that can make an incision in accordance with the bead, particle, and/or marker dimensions to be placed at an intended depth. In some embodiments the depth may be between about 5 and about 300 microns. In some embodiments the depth may be between about 50 to about 250 microns. In some other embodiments, the depth may be between about 150 to about 200 microns. In some embodiments, the depth can be greater than about 5 microns, greater than about 45 microns, greater than about 85 microns, greater than about 125 microns, greater than about 165 microns, greater than about 205 microns, greater than about 245 microns, greater than about 285 microns, or about greater than about 300 microns. In some cases, the depth can be less than about 5 microns, less than about 45 microns, less than about 85 microns, less than about 125 microns, less than about 165 microns, less than about 205 microns, less than about 245 microns, less than about 285 microns, or less than about 300 microns

The incision may be made with a microsurgical knife 303 in a horizontal setting, and the beads, particles, and/or markers may be delivered to the implantation area via a guide 301. The implantation guide 301 may carry one or more fluorescent beads, one or more particles, and/or one or more markers 304 and can deliver them into the pocket. In some embodiments, the guide may have a silicon tip that may assist with performing a corneal incision.

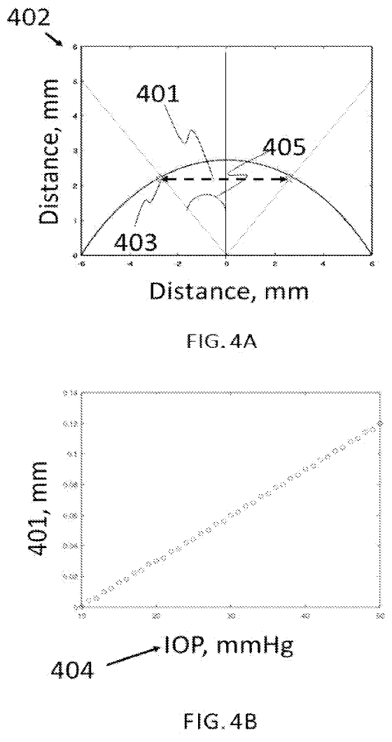

Referring to FIG. 4A, a cross-section of a model cornea 402 is shown with two fluorescent microparticle and/or marker implants 403. In some embodiments, each fluorescent particle and/or marker may be placed into a surgically created pocket. Each pocket may be shaped to hold the particle and/or marker in positions, and prevent the particle and/or marker from drifting, moving, or otherwise migrating. The pocket may be sealed or otherwise treated so as to reinforce the native tissue to prevent particle and/or marker migration. This reinforcement may be the addition of additional nutrients to accelerate healing of the cornea, growth factors, or some artificial material that may strengthen the pocket.

In various embodiments, the observed distance between the implants 401 may be measure in mm, or as a ratio of the length of the distance between two or more pairs of implants (e.g., particles and/or markers), or the measurement from one particle and/or marker to an artificial fiducial (a fixed position implant/particle) or a natural fiducial (the center of the eye, the position of a known structure, and so on). In the various embodiments, the distance (real or observed) between implants (or implant and fiducial) may be measured to help determine the intraocular pressure within the eye.

In some embodiments, the angle 405 of the implant of the axis of vision (or the cylindrical axis of symmetry, e.g., the optical axis of the eye) as measured from the central axis of the pupil may be used to help determine the optimal positioning of the fluorescent beads, particles, and/or markers, where their separation is most sensitive to the changes in IOP. The determination of the angle is mathematically equivalent to the selection of the radius at which the fluorescent particles and/or markers are implanted.

Now referring to FIG. 4B, a calculation of the distance between implants 401 as a function of IOP 404 is given. The corneal surface along the XZ plane can be described according to the relation between the instantaneous tangential radius of curvature ρ(x) and asphericity Q can be derived as follows. In the vertical (y) meridian the conic section is expressed by:

$${x}^{2} + ( 1 + Q ) {z}^{2} - 2 z R = 0 \qquad ( Eq . 1 )$$

where Q and R are the asphericity and radius of curvature of the cornea surface, respectively. Based on Eq.1, and assuming about a 4-micrometer displacement of the corneal apex upon about 1 mmHG IOP change, and also assuming the scleral-corneal interface is not affected by IOP change, the relative distance between the two implants can be calculated. The calculation assumes that the edges of the cornea where it meets the sclera are fixed and the cornea bulges in response to an increase in the IOP. This perturbation is assumed to be first order, to affect the radius of curvature, and to not the aspheric parameter Q. In order to estimate the cornea surface position at a given IOP for a corneal aperture of X=−6 to 6 mm (assuming a corneal size of 12 mm in total), we assume a radius of curvature that is given by R(IOP)=R(IOP_{ref})−(IOP−IOP_{ref})*0.004 mm. The z position of the corneal surface is calculated through solving Eq.1 numerically and the minimum displacement at the edges (zₘᵢₙ) of the cornea are found for x=−6 and whole z(x) curve is shifted by subtracting the minimum of the corneal position zₘᵢₙ. This is to emulate the assumption that the sclera/cornea interface does not change height upon a change in the IOP. The resulting corneal position is then used to calculate the x and z displacements of an implant. Assuming, to first order approximation, that the angle between the implanted fluorescent particle and/or marker and origin of the cornea (e.g., the intersection point of the sclera/cornea interface plane and central axis or the cornea) does not change, the x and z positions of the implant are calculated for the implants for the reference IOP and actual IOP. The change in the distance between two implants can then be calculated by taking the differences of their x positions. The distance between two implants that are symmetrically positioned on opposite sides of the cornea optical axis are calculated. The results of such a calculation is plotted as a function of IOP, for 45-degree implant angles. The procedure thus converts the change in the radius of curvature (which can be difficult to precisely measure using a simple front facing camera) into a displacement in the imaging field, making it possible to precisely determine relative positions of various implants on the cornea.

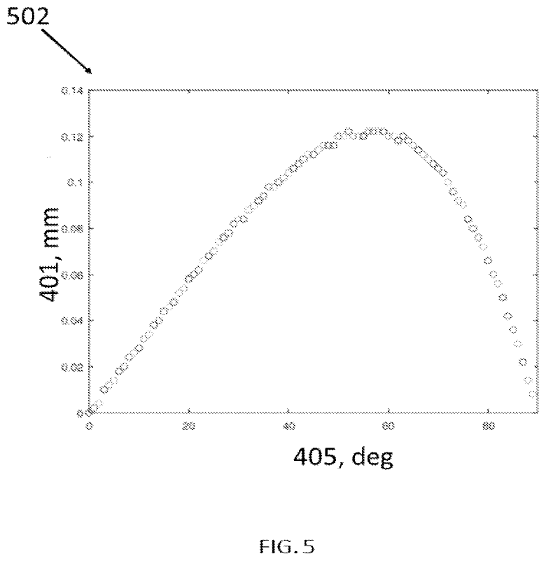

In some embodiments, the change in distance between two symmetrically (symmetrical relative to the optical axis of the cornea, or z-axis) positioned implants across the cornea as a function of positioning angle 502 is shown in FIG. 5 for an about 40 mmHg IOP change. The distance change may depend on the angle of implantation (or equivalently the radius at which the fluorescent particles and/or markers are implanted), and may be maximized at e.g., about 50-60 degrees. In some cases, the distance change can be maximized for about 45-65 degrees. This angle may correspond to about 60-70 percent of the radius of the cornea based on geometric calculations done on the schematic eye model. As the angle is increased, the displacement may decrease. For example, for an angle of about 85 degrees, the implants may be at about 90% radius of the full corneal radius, and the displacement may be halved. By implanting four fluorescent particles and/or markers, one pair at ±90% radius and second pair at ±60% radius, one can achieve an imaging configuration which may be insensitive to changes in camera distance and eye rotation (for small deviations from perfectly centered eye). Alternatively, by implanting four fluorescent particles, one pair at ±30% radius (where they are closer to the apex of cornea, but are far enough from the pupil, so as not to block the vision) and second pair at ±60% radius, one can achieve an imaging configuration which may be insensitive to changes in camera distance and eye rotation (for small deviations from perfectly centered eye). The measurement may be corrected for a person's eye taking into account the ophthalmologically determined data measured at a reference pressure, such as radius of curvature and/or aspheric parameter Q. By taking the ratio of the distance between the outer implants (e.g., the implants closer to the sclera) and inner implants (e.g., that are at about 60% of the corneal radius) a normalized displacement may be calculated. The normalized displacement may not be sensitive to imaging magnification or small eyeball rotations. In some embodiments, this ratio may be used to calculate the IOP using the reference initial measurement taken at a known IOP. One or more cameras positioned to view the eye may be used to further refine this measurement. The measurement of the corneal apex position and implant height with respect to one or more cameras may be used to compensate for magnification errors and eye rotation. Off angle views from one or more cameras may be used to make a correction in the measured displacement values. Such off-angle views may be from the side, top, bottom, other angle of about 60 degrees off the main axis of the eye (the viewing axis of the person looking directly ahead), or any combination thereof.

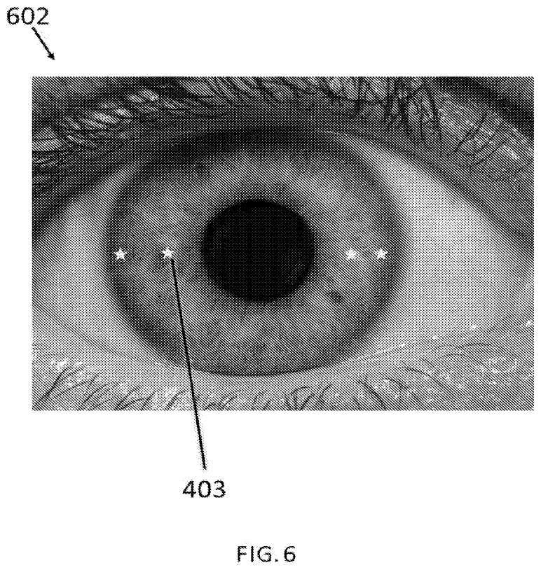

In some embodiments, referring to FIG. 6, there is an image of an eye 602. In some embodiments, a camera facing the front of the eye may use a live image, a captured image, or a combination thereof, to determine the position of the eye features (e.g., the iris, pupil, cornea, a combination thereof, and so on) as well as any particles and/or markers placed into the eye. In various embodiments as described herein, particles and/or markers 403 may be placed into pockets at one or more depths in the cornea, and/or other surface structures of the eye such as the sclera, the sclera-corneal interface, or a combination thereof. The particles and/or markers in these pockets may be visible to the front facing camera and seen in the image 602. The stars in the image are representations of the particles and/or markers placed into the surface of the eye. In some embodiments, the particles and/or markers may be placed along an x and y axis, where the origin may be an imaginary point in the center of the pupil of an eye. The two particles and/or markers in the positive x direction, and the two particles and/or markers in the negative x direction, may form a pair of inner and outer implants, as discussed elsewhere herein. The observed difference in position, and/or calculated difference in some ratio between these points, may be used to determine the IOP of the eye.

In some cases, the markers may be embedded on a surface of the eye, in the cornea of the eye, in the lens of the eye, in a tissue adjacent to the eye, or any combination thereof. The markers can be fluorescent, as described herein. The markers can be positioned at a distance of up to about 10 mm from each other. The markers can be positioned at a distance of about 0.5 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, or about 10 mm from each other. The markers may be positioned at a distance of up to about 0.5 mm, up to about 1 mm, up to about 1.5 mm, up to about 2 mm, up to about 2.5 mm, up to about 3 mm, up to about 3.5 mm, up to about 4 mm, up to about 4.5 mm, up to about 5 mm, up to about 5.5 mm, up to about 6 mm, up to about 6.5 mm, up to about 7 mm, up to about 7.5 mm, up to about 8 mm, up to about 8.5 mm, up to about 9 mm, up to about 9.5 mm, or up to about 10 mm from each other. The markers may be positioned at a distance of at least about 0.5 mm, at least about 1 mm, at least about 1.5 mm, at least about 2 mm, at least about 2.5 mm, at least about 3 mm, at least about 3.5 mm, at least about 4 mm, at least about 4.5 mm, at least about 5 mm, at least about 5.5 mm, at least about 6 mm, at least about 6.5 mm, at least about 7 mm, at least about 7.5 mm, at least about 8 mm, at least about 8.5 mm, at least about 9 mm, at least about 9.5 mm, or at least about 10 mm from each other.

In some cases, there may be multiple pairs of markers. In some cases, there may be about 2, about 3, or about 4 pairs of markers. In some cases, there may be up to about 2, up to about 3, or up to about 4 pairs of markers. In some cases, there may be at least about 2, at least about 3, or at least about 4 markers. The TOP of the eye can be determined from a ratio of a distance between a first marker and a second marker of a first pair of markers and a distance between a third marker and a fourth marker of a second pair of markers. The IOP of the eye can be determined from a ratio between additional pairs of markers.

In some cases, the fluorescent bead, particle, and/or marker 403 can be annular, tubular, circular, elliptical, cylindrical, helical, hexagonal, triangular, square-shaped, rectangular, quadrilateral, or any other shape.

In some cases, the diameter of the fluorescent particle 403 can be between about 10 microns to about 1000 microns. The diameter of the fluorescent particle and/or marker can be between about 10 microns to about 100 microns, about 10 microns to about 200 microns, about 10 microns to about 300 microns, about 10 microns to about 400 microns, about 10 microns to about 500 microns, about 10 microns to about 600 microns, about 10 microns to about 700 microns, about 10 microns to about 800 microns, about 10 microns to about 900 microns, about 10 microns to about 1000 microns, about 100 microns to about 200 microns, about 100 microns to about 300 microns, about 100 microns to about 400 microns, about 100 microns to about 500 microns, about 100 microns to about 600 microns, about 100 microns to about 700 microns, about 100 microns to about 800 microns, about 100 microns to about 900 microns, about 100 microns to about 1000 microns, about 200 microns to about 300 microns, about 200 microns to about 400 microns, about 200 microns to about 500 microns, about 200 microns to about 600 microns, about 200 microns to about 700 microns, about 200 microns to about 800 microns, about 200 microns to about 900 microns, about 200 microns to about 1000 microns, about 300 microns to about 400 microns, about 300 microns to about 500 microns, about 300 microns to about 600 microns, about 300 microns to about 700 microns, about 300 microns to about 800 microns, about 300 microns to about 900 microns, about 300 microns to about 1000 microns, about 400 microns to about 500 microns, about 400 microns to about 600 microns, about 400 microns to about 700 microns, about 400 microns to about 800 microns, about 400 microns to about 900 microns, about 400 microns to about 1000 microns, about 500 microns to about 600 microns, about 500 microns to about 700 microns, about 500 microns to about 800 microns, about 500 microns to about 900 microns, about 500 microns to about 1000 microns, about 600 microns to about 700 microns, about 600 microns to about 800 microns, about 600 microns to about 900 microns, about 600 microns to about 1000 microns, about 700 microns to about 800 microns, about 700 microns to about 900 microns, about 700 microns to about 1000 microns, about 800 microns to about 900 microns, about 800 microns to about 1000 microns, and between about 900 microns to about 1000 microns.

In some cases, the diameter of the fluorescent particles and/or markers can be less than about 10 microns, less than about 100 microns, less than about 200 microns, less than about 300 microns, less than about 400 microns, less than about 500 microns, less than about 600 microns, less than about 700 microns, less than about 800 microns, less than about 900 microns, or less than about 1000 microns. In some cases, the diameter of the fluorescent particles and/or markers can be greater than about 10 microns, greater than about 100 microns, greater than about 200 microns, greater than about 300 microns, greater than about 400 microns, greater than about 500 microns, greater than about 600 microns, greater than about 700 microns, greater than about 800 microns, greater than about 900 microns, or greater than about 1000 microns.

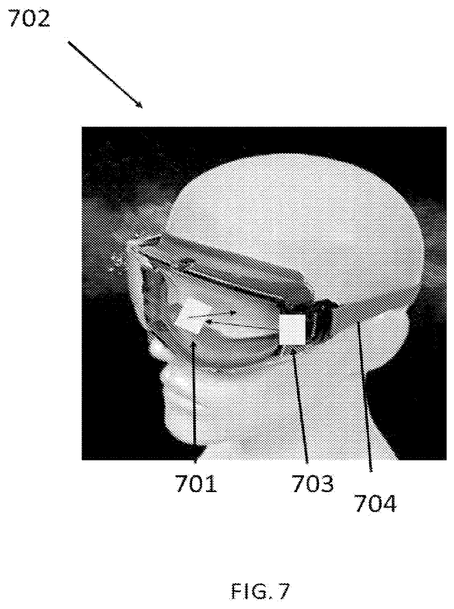

Referring to FIG. 7, an example eyewear, e.g., a goggle 702 with a pair of cameras is shown according to an embodiment described herein. A front camera 701 and a side camera 703 may be aimed generally at the surface of the eye, such that the camera(s) may view the eye and/or capture one or more images of the eye. In some embodiments, a front camera may be generally positioned on the visual axis of the eye and be looking “down” on the eye capturing an image of the eye from a front perspective view of the eye. In some embodiments, there may be a side camera that may view the eye from an off angle. The off-angle view may be to the side as shown in FIG. 7, or at some other angle where an image may be captured or viewed. The arrows aiming towards the eye from the boxes, as shown in FIG. 7, represent the lines of sight of the cameras.

The goggles can comprise a chassis, and/or frame, and one or more lens. The chassis can at least partially contain one or more detectors, a light source, or a combination thereof. The chassis can comprise eyewear, eyeglasses, goggles, heads up display, virtual reality eyewear, augmented reality eyewear, or any combination thereof.

In some cases, the goggles can be full coverage goggles 702, as shown in FIG. 7. In some cases, the goggles can be slimmer. In some cases, the thickness of the goggles can be between about 10 mm to about 2 mm. In some cases, the thickness of the goggles can be between about 2 mm to about 4 mm, about 2 mm to about 6 mm, about 2 mm to about 8 mm, about 2 mm to about 10 mm, about 4 mm to about 6 mm, about 4 mm to about 8 mm, about 4 mm to about 10 mm, about 6 mm to about 8 mm, about 6 mm to about 10 mm, or between about 8 mm to about 10 mm. In some cases, the thickness of the goggles can be less than about 10 mm, less than about 8 mm, less than about 6 mm, less than about 4 mm, or less than about 2 mm.

In some cases, the goggles can be without the top, bottom, or both portions of the goggles that contact a subject's face. In some cases, the goggles may comprise glasses. In some cases, the goggles may comprise laboratory glasses. The goggles may comprise shaded glasses (e.g., sunglasses). The goggles can have a tinting function similar to transition glasses. The goggles may comprise indoor glasses.

In some cases, the goggles can have a band 704 around the head, as shown in FIG. 7. The goggles can have ear loops or temple tips, e.g., to hold the weight of the goggles with the cameras such that the cameras may be stabilized when taking one or more images.

The goggles may be any sort of eyewear goggles used for modern eyewear, including goggles for sunglasses, vision correction glasses, safety glasses, goggles of all types (e.g., Swimming, athletic, safety, skiing, and so on). The goggles may be suitable for a single lens for one eye, a lens for two eyes (e.g., a visor), a single lens and an eye cover (such as for persons with amblyopia or who may suffer from the loss of one eye), or any combination thereof. The lens may comprise one or more prescription lenses for vision correction, a clear or tinted lens for appearance, an opaque lens that covers the eye, or any combination thereof. In some embodiments, the lens may comprise a defined area for the field of view of the eye of the user. The field of view may be clear to avoid blocking the vision of the eye(s) of the user. The various elements of the eyewear device may be place on the periphery of the lens, on the frame, or a combination thereof. The goggles may have flanges, other protrusions, tabs, or a combination thereof, for the attachment of image sensors, light sources, battery, computational devices, any other component suitable for the use with the present disclosure, or any combination thereof.

The goggles can be positioned at a distance up to about 2 centimeters (cm) from a tangential surface of the eye. The goggles can be positioned at a distance of at least about 0.5 cm, at least about 1 cm, at least about 1.5 cm, or at least about 2 cm. In some cases, the goggles can be positioned at a distance of less than about 0.5 cm, less than about 1 cm, less than about 1.5 cm, or less than about 2 cm.

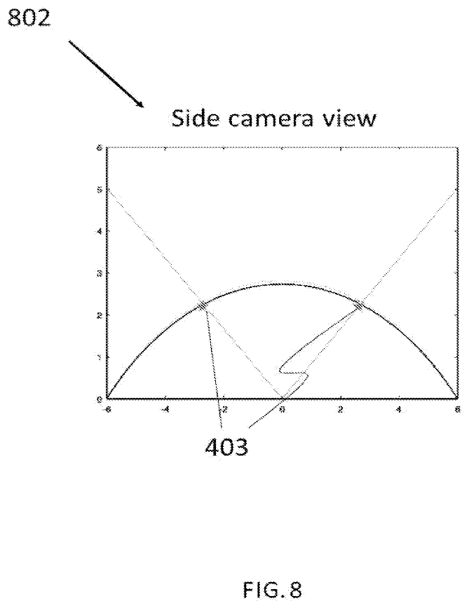

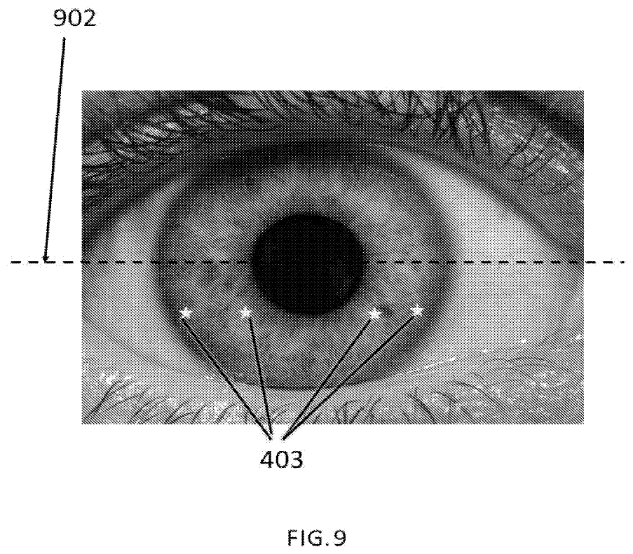

Referring to FIG. 8, a schematic of the view of a side view camera 802, described elsewhere herein, is shown. The side view image and/or off angle image acquired, detected, and/or obtained by the side view camera may show the position of one or more particles and/or markers 403 (e.g., implants, as described elsewhere herein) and their relative angle off the central axis of the eye. In some cases, the side view image and/or off angle image may show two or more particles 403. In some cases, the side view image and/or off angle image may show four particles 403, as shown in FIG. 9. An artificial origin may be used to determine an angle from the main axis of the eye, described elsewhere herein, for each particle. The image may show a stationary fiducial, which may be another implant particle and/or marker, an anatomical feature, an artificial reference point (e.g., as may be attached to the goggle), or any combination thereof.

In some embodiments, the positions of the implanted fluorescent particle(s) and/or marker(s) may be displaced from the center of the cornea 902, as shown in FIG. 9. In some embodiments, the position of the particles 403 may not be aligned to an artificial x or y axis. The particles may be in any orientation or alignment so long as their positions can be accurately measured, whether for actual distances and/or observed distances.

In some cases, the markers are embedded on a surface of the eye, in the cornea of the eye, in the lens of the eye, in a tissue adjacent to the eye, or any combination thereof. The markers can be fluorescent, as described herein. The markers can be positioned at a distance of up to about 10 mm from each other. The markers can be positioned at a distance of about 0.5 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, or about 10 mm from each other. The markers may be positioned at a distance of up to about 0.5 mm, up to about 1 mm, up to about 1.5 mm, up to about 2 mm, up to about 2.5 mm, up to about 3 mm, up to about 3.5 mm, up to about 4 mm, up to about 4.5 mm, up to about 5 mm, up to about 5.5 mm, up to about 6 mm, up to about 6.5 mm, up to about 7 mm, up to about 7.5 mm, up to about 8 mm, up to about 8.5 mm, up to about 9 mm, up to about 9.5 mm, or up to about 10 mm from each other. The markers may be positioned at a distance of at least about 0.5 mm, at least about 1 mm, at least about 1.5 mm, at least about 2 mm, at least about 2.5 mm, at least about 3 mm, at least about 3.5 mm, at least about 4 mm, at least about 4.5 mm, at least about 5 mm, at least about 5.5 mm, at least about 6 mm, at least about 6.5 mm, at least about 7 mm, at least about 7.5 mm, at least about 8 mm, at least about 8.5 mm, at least about 9 mm, at least about 9.5 mm, or at least about 10 mm from each other.

In some cases, there are multiple pairs of markers. In some cases, there are about 2, about 3, or about 4 pairs of markers. In some cases, there are up to about 2, up to about 3, or up to about 4 pairs of markers. In some cases, there are at least about 2, at least about 3, or at least about 4 markers. The IOP of the eye can be determined from a ratio of a distance between a first marker and a second marker of a first pair of markers and a distance between a third marker and a fourth marker of a second pair of markers. The IOP of the eye can be determined from a ratio between additional pairs of markers.

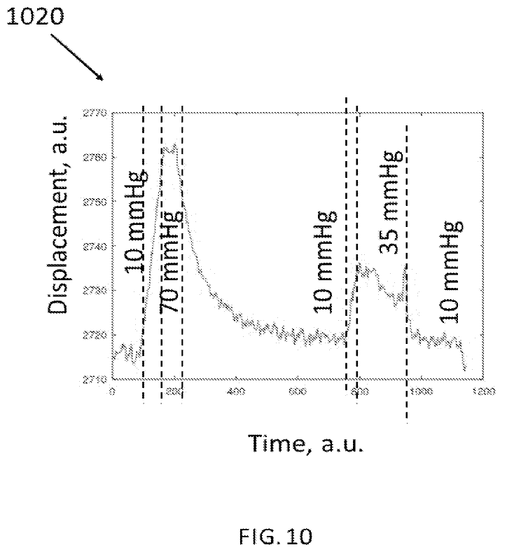

In some embodiments, an artificial eye model made out of elastomeric material can be used to measure the distance between embedded fluorescent microparticles and/or markers as a function of applied IOP. The data shown in FIG. 10 shows the correlation between the microparticle displacements and applied IOP 1020. The positions of the particles may be measured on the captured one or more images obtained and/or detected by a front facing camera, a side viewing camera, or a combination thereof, of a model eye, and central peaks of the spots corresponding to the fluorescent beads and/or markers may be calculated using a center-of-mass calculation or peak fitting to the gaussian intensity profiles of the spots. Higher IOP values may result in an increase of the distance between the fluorescent beads and/or markers. The fluorescent implants may be assumed to have a narrowband excitation spectrum (e.g., green light as a non-limiting example, may be used to excite) and narrow band emission spectrum (e.g., red light as a non-limiting example, may be emitted). In practice, a fluorescence imaging camera may be used, where a fluorescence excitation light source (e.g., a green LED) and a normal illumination source (e.g., a red LED) are sequentially illuminating the one or more particles and/or one or more markers as one or more images may be collected, detected, and/or obtained for red and/or green illuminations. One or more images captured after illumination with the red illuminator may provide a monochrome regular image of the eye, allowing the neural network to find and/or identify the cornea position, based on e.g., pattern matching. The green illuminator may be blocked by the emission filter in front of the camera and only the dye doped fluorescent particles and/or markers may be imaged as bright spots, when the green LED is on, eliminating the background from the iris, and/or the rest of the eye. The monochrome regular image collected at an earlier time point (e.g., 30 msec ahead of the image of the one or more particles and/or markers illuminated by the green illuminator) when the eye is illuminated by the red illuminator may be used as a guide to estimate the vicinity of the positions of the fluorescent beads and/or markers. In some cases, an area based on the monochrome regular image may be used to define windows for curve fitting or center-of-mass calculations to precisely determine the peak positions of the fluorescent bead and/or marker spots. This way, unwanted residual fluorescence from the iris and other eye tissue noise sources may be excluded and a precise position of the particles and/or markers may be obtained.

In some cases, unwanted background noise of residual fluorescence can be reduced by between about 5% and about 95%. In some cases, unwanted background noise of residual fluorescence can be reduced by at least about 5%, at least about 15%, at least about 25%, at least about 35%, at least about 45%, at least about 55%, at least about 65%, at least about 75%, at least about 85%, or at least about 95%. In some cases, unwanted background noise of residual fluorescence can be reduced by less than about 5%, less than about 15%, less than about 25%, less than about 35%, less than about 45%, less than about 55%, less than about 65%, less than about 75%, less than about 85%, or less than about 95%.

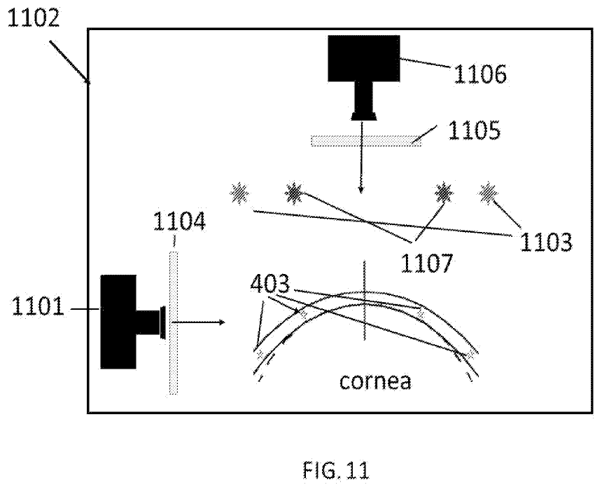

In some embodiments, a schematic showing a sample cross section of the imaging system 1102 is shown in FIG. 11. This system and the devices described within the system can be used in methods, described elsewhere herein, to measure IOP of an eye.

Using one or more cameras, a user can obtain and/or receive a first image. The image can be of at least two particles and/or markers embedded in the eye. The image can be detected with a first detector. The first detector can be a camera, a charge coupled device (CCD) sensor, complementary metal oxide semiconductor (CMOS) sensor, or any combination thereof. The detector can be configured to detect a signal of at least two particles and/or markers embedded in the eye. The image can be taken by cameras disposed on goggles, glasses, other eyewear, or any combination thereof. Using one or more cameras, a user can obtain or receive a second image. The second image can be of the at least two particles and/or markers embedded in the eye. The image can be detected with a second detector. The second detector can be a camera, a charge coupled device (CCD) sensor, complementary metal oxide semiconductor (CMOS) sensor, or any combination thereof. The second detector can be a different camera than the camera of the first detector. One or more detectors can be contained at least partially in the goggles, as described elsewhere herein. An optical axis of the first detector can be at an angle with respect to an optical axis of the second detector. A user can determine the IOP of the subject with embedded particles and/or markers from a position of one or more of the at least two particles and/or markers from a first image or one or more of the at least two markers from a second image. In some cases, the position of the at least two markers and/or particles can be provided as an input to a machine learning algorithm or a predictive model trained to provide an output related to the TOP of the eye.

In some cases, the markers are embedded on a surface of the eye, in the cornea of the eye, in the lens of the eye, in a tissue adjacent to the eye, or any combination thereof. The markers can be fluorescent, as described herein. The markers can be positioned at a distance of up to about 10 mm from each other. The markers can be positioned at a distance of about 0.5 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, or about 10 mm from each other. The markers may be positioned at a distance of up to about 0.5 mm, up to about 1 mm, up to about 1.5 mm, up to about 2 mm, up to about 2.5 mm, up to about 3 mm, up to about 3.5 mm, up to about 4 mm, up to about 4.5 mm, up to about 5 mm, up to about 5.5 mm, up to about 6 mm, up to about 6.5 mm, up to about 7 mm, up to about 7.5 mm, up to about 8 mm, up to about 8.5 mm, up to about 9 mm, up to about 9.5 mm, or up to about 10 mm from each other. The markers may be positioned at a distance of at least about 0.5 mm, at least about 1 mm, at least about 1.5 mm, at least about 2 mm, at least about 2.5 mm, at least about 3 mm, at least about 3.5 mm, at least about 4 mm, at least about 4.5 mm, at least about 5 mm, at least about 5.5 mm, at least about 6 mm, at least about 6.5 mm, at least about 7 mm, at least about 7.5 mm, at least about 8 mm, at least about 8.5 mm, at least about 9 mm, at least about 9.5 mm, or at least about 10 mm from each other.

In some cases, there may be multiple pairs of markers. In some cases, there may be about 2, about 3, or about 4 pairs of markers. In some cases, there may be up to about 2, up to about 3, or up to about 4 pairs of markers. In some cases, there may be at least about 2, at least about 3, or at least about 4 markers. The IOP of the eye can be determined from a ratio of a distance between a first marker and a second marker of a first pair of markers and a distance between a third marker and a fourth marker of a second pair of markers. The IOP of the eye can be determined from a ratio between additional pairs of markers.

In some cases, a light source is provided to the markers embedded in the eye to obtain and/or detect the first or second image, described elsewhere herein. There can be one or more light sources. The light source can comprise a light emitting diode (LED), laser, broad band super luminescent diode, coherent light source, or any combination thereof. The light source can comprise a LED. The light emitting diode can emit light at a variety of bands of wavelengths of the visual spectrum, e.g., from a green wavelength to a red wavelength. The light source can be located on the goggles as described above. The light source can be optically coupled to one or more eye(s) of the subject.

In some cases, one or more polarizing filters may be used to obtain one or more images with the first detector and/or the second detector. The first image of the at least two particles and/or markers embedded in the eye can be obtained or detected by the first detector through a first polarizing filter. The second image of the at least two particles and/or markers embedded in the eye can be obtained or detected by a second detector through a second polarizing filter.

In some cases, one or more processors may be electrically coupled to one or more detectors. The processors can be configured to process the signal of the two or more particles and/or markers embedded in the eye and determine the IOP of the eye from a position of the two or more particles and/or markers. The processor can comprise a memory. The memory can be separate from the processor. In some cases, the processors may be external to the system shown in FIG. 11. The processor can be located on an eyewear device, a remote processing device, a remote server, a cloud server, or any combination thereof. The processors can obtain or receive one or more images from a server, cloud base storage, eyewear device, or any combination thereof.

The memory can store one or more programs for execution by the one or more processors. The one or more programs can comprise instructions to obtain and/or receive the results of the imaging system, described elsewhere herein. The one or more programs can comprise instructions to obtain and/or receive one or more images of at least two particles and/or markers embedded in the eye. The one or more images can be detected with a first detector and/or a second detector. An optical axis of the first detector can be at an angle with respect to an optical axis of the second detector, as described elsewhere herein. The program can comprise instructions to determine the IOP of the eye from a distance between the at least two particles and/or markers in the one or more images.

Referring specifically to an example system as shown in FIG. 11, the front facing camera 1106 (e.g., a first detector, described elsewhere herein) may be placed behind a long pass optical filter 1105 with a yellow (e.g., about 580 nm cutoff wavelength) positioned to acquired and/or obtain one or more plane view images of the eye with implanted particles 403. The side facing camera 1101 (e.g., a second detector, described elsewhere herein) may also be placed behind a long pass optical filter 1104 with a yellow (about 580 nm cutoff wavelength). One or more fluorescence excitation light sources 1103 (e.g., a green LED, about 520 nm-540 nm emission wavelength) may provide excitation light to one or more microparticle and/or marker implants. A regular imaging illuminator 1107 (e.g., red LED, about 650 nm center wavelength emission) may be turned off when the fluorescence excitation light source 1103 is turned on and/or emitting light. After acquiring the fluorescent image, the green LED 1103 may be turned off and one or more red LEDs 1107 may be turned on, to take, acquire, detect, and/or obtain a regular image of the eye identifying one or more eye anatomical features (e.g., iris, sclera, sclera-cornea boundary, or any combination thereof). The brightfield image taken with red illumination may be used to position the pupil, the iris, the cornea, or any combination thereof, which may provide additional information about the positioning of the cornea with respect to the first detector, second detector, regular imaging illuminator, fluorescent imaging illuminator, or any combination thereof. The fluorescent image may then be used to extract the positions of the microparticles, particles, and/or makers embedded in the eye by fitting gaussian functions to the particle and/or maker images. Accurate determination of the peak positions of the particle and/or marker blob representations corresponding to the microparticles and/or markers can be achieved with Gaussian fitting. Sub-pixel accuracy, provided by Gaussian fitting, may allow precise determination of inter-particle and/or marker distances.

A front camera 1106 and/or a side viewing camera 1101 may be directed and/or aligned at a surface of the eye, such that the front camera 1106 and/or the side view camera 1101 may view the eye and/or capture images of particles and/or makers implanted and/or embedded in the eye. In some embodiments, a front camera 1106 may be positioned on the visual axis, e.g., looking “down” on the eye and viewing the eye from directly in front of the eye. In some embodiments, there may be a side viewing camera 1101 that may view the eye from an off angle. The off angle view may be to the side as shown in FIG. 11, or at some other angle where one or more images may be captured, obtained, and/or viewed. The arrows aiming towards the eye from the boxes represent the lines of sight of the cameras, as shown in FIG. 11.

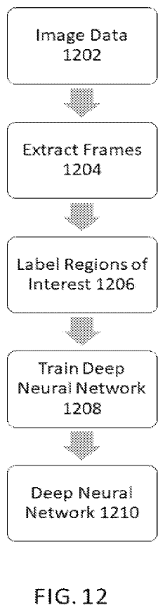

In some embodiments, image data 1202 may be used to train a deep neural network 1210 in a process illustrated in FIG. 12.

In some cases, one or more processors may be electrically coupled to one or more detectors (e.g., the first and/or second detector, described elsewhere herein). In some cases, one or more processors may be electrically coupled to a system as described herein. The processors can be configured to process the signal of the two or more particles and/or markers embedded in the eye and determine the IOP of the eye from a position of the two or more particles and/or markers. The processor can be located on an eyewear device, a remote processing device, a remote server, a cloud server, or any combination thereof. The processors can obtain and/or receive one or more images from a server, cloud base storage, eyewear device, or any combination thereof. The processor can comprise a memory. The memory can be separate from the processor.

The memory can store one or more programs for execution by the one or more processors. The one or more programs can comprise instructions to obtain and/or receive the results of the imaging system described above. The one or more programs can comprise instructions to obtain and/or receive one or more images of at least two particles and/or markers embedded in the eye. The one or more images can be detected with a first detector and/or a second detector, as described elsewhere herein. An optical axis of the first detector can be at an angle with respect to an optical axis of the second detector, as described elsewhere herein. The program can comprise instructions to determine the IOP of the eye from a distance between the at least two markers in the one or more images.

With reference to FIG. 12, the position of at least two markers comprising image data 1202 can be provided as an input to a machine learning algorithm or a predictive model trained to provide an output related to the TOP of the eye. The image data 1202 may be one or more still image and/or one or more video frame, segments, and/or clips from any suitable source, such as one or more cameras and/or data storage medium (e.g., memory). The machine learning algorithm(s) and/or predictive model(s) can receive and/or obtain one or more images of at least two particles and/or markers embedded in one or more eyes and a corresponding IOP of the one or more eyes through systems described herein. An untrained and/or partially untrained machine learning algorithm and/or predictive model can be trained with the one or more images of the at least two particles and/or markers embedded in the one or more eyes and the corresponding IOP of the one or more eyes to produce a trained machine learning algorithm and/or trained predictive model. The untrained or partially untrained machine learning algorithm and/or predictive model can also be trained on a distance between the at least two particles and/or markers in the one or more images of the at least two particles and/or markers embedded in the one or more eyes.

The training process may comprise extracting one or more frames 1204 from the image data 1202 where various regions may be identified and/or labeled to produce one or more label regions of interest 1206. The label regions of interest 1206 may number in the tens, hundreds, or thousands, and may be drawn from a similar number of image data 1202 sources. The label regions of interest 1206 of the image data 1202 may be considered as the training data to train deep neural network 1208. Once the training of the deep neural network 1208 is deemed sufficient, based on an acceptable error rate of new data inputs compared to analysis by a trained individual, the deep neural network 1210 may be trained and ready for diagnostic application with subjects' data not used to train the machine learning algorithm and/or predictive model.

An acceptable error rate may be less than about 10%. An acceptable error rate may be less than about 10%, less than about 8%, less than about 6%, less than about 4%, less than about 2%, less than about 1%, less than about 0.5%, or less than about 0.1%. An acceptable error rate of calculated position and/or of the one or more particles and/or one or more markers and/or a distance between the one or more particles and/or one or more markers may be between about 0.1 mm to about 0.5 mm, about 0.1 mm to about 1 mm, about 0.1 mm to about 2 mm, about 0.1 mm to about 4 mm, about 0.1 mm to about 6 mm, about 0.1 mm to about 8 mm, about 0.1 mm to about 10 mm, about 0.5 mm to about 1 mm, about 0.5 mm to about 2 mm, about 0.5 mm to about 4 mm, about 0.5 mm to about 6 mm, about 0.5 mm to about 8 mm, about 0.5 mm to about 10 mm, about 1 mm to about 2 mm, about 1 mm to about 4 mm, about 1 mm to about 6 mm, about 1 mm to about 8 mm, about 1 mm to about 10 mm, about 2 mm to about 4 mm, about 2 mm to about 6 mm, about 2 mm to about 8 mm, about 2 mm to about 10 mm, about 4 mm to about 6 mm, about 4 mm to about 8 mm, about 4 mm to about 10 mm, about 6 mm to about 8 mm, about 6 mm to about 10 mm, and between about 8 mm to about 10 mm.

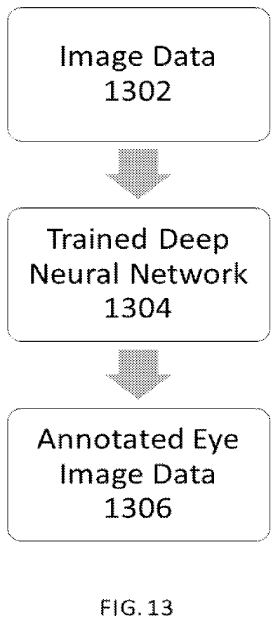

In some embodiments, the operation of using the deep neural network with image data from a patient is illustrated in FIG. 13.

In some cases, one or more processors may be electrically coupled to one or more detectors. In some cases, one or more processors may be electrically coupled to a system as described herein. The processors can be configured to process the signal of the two or more particles and/or markers embedded in the eye and determine the IOP of the eye from a position of the two or more markers. The processor can be located on an eyewear device, a remote processing device, a remote server, a cloud server, or any combination thereof. The processors can obtain and/or receive one or more images from a server, cloud base storage, eyewear device, or any combination thereof. The processor can comprise a memory. The memory can be separate from the processor.

The memory can store one or more programs for execution by the one or more processors. The one or more programs can comprise instructions to obtain and/or receive the results of the imaging system described above. The one or more programs can comprise instructions to obtain and/or receive one or more images of at least two particles and/or markers embedded in the eye. The one or more images can be detected with a first detector and/or a second detector. An optical axis of the first detector can be at an angle with respect to an optical axis of the second detector, as described elsewhere herein. The program can comprise instructions to determine the TOP of the eye from a distance between the at least two particles and/or markers in the one or more images.

Referring to FIG. 13, the image data 1302 may be drawn from one or more cameras, a memory device, from an intermediate data source, such as the cloud, a data bus, processor, in a camera memory, computing device memory, or any combination thereof. The received image data 1302 may be input into the trained deep neural network 1304, where the trained deep neural network machine learning algorithm and/or trained predictive model may produce an annotated eye image data set 1306, which can be displayed to a screen (mobile phone, tablet, computer screen, or any combination thereof) and/or stored to a computing device memory for later retrieval. The deep neural network 1304 can be trained as described elsewhere herein, or via additional methods.

By using the process provided, a subject who may be monitoring their IOP, may have speedy and reliable results using the systems, devices, and methods described herein in combination with the apparatus for measuring IOP from the placement of the fluorescent beads, particles, and/or markers.

### Examples of Electronic Components

Embodiments of the subject matter and the operations described in this specification may be implemented in digital electronic circuitry, or in computer software, firmware, and/or hardware, including the structures disclosed in this disclosure and their structural equivalents, or any combination thereof. Embodiments of the subject matter described in this specification may be implemented as one or more computer programs, e.g., one or more modules of computer program instructions, encoded on one or more computer storage medium for execution by, or to control the operation of, data processing apparatus, such as a processing circuit. A controller and/or processing circuit such as CPU may comprise any digital and/or analog circuit components configured to perform the functions described herein, such as a microprocessor, microcontroller, application-specific integrated circuit, programmable logic, etc., or any combination thereof. In some cases, the program instructions may be encoded on an artificially generated propagated signal, e.g., a machine-generated electrical, optical, and/or electromagnetic signal, that may be generated to encode information for transmission to suitable receiver apparatus for execution by a data processing apparatus.

A computer storage medium may be, or be included in, a computer-readable storage device, a computer-readable storage substrate, a random or serial access memory array or device, or any combination thereof. Moreover, while a computer storage medium is not a propagated signal, a computer storage medium may be a source or destination of computer program instructions encoded in an artificially generated propagated signal. The computer storage medium may also be, or be included in, one or more separate components or media (e.g., multiple CDs, disks, other storage devices, or any combination thereof). Accordingly, the computer storage medium can be both tangible and non-transitory.

The operations described in this disclosure may be implemented as operations performed by a data processing apparatus on data stored on one or more computer-readable storage devices and/or received from other sources. The term “data processing apparatus” or “computing device” encompasses all kinds of apparatus, devices, and/or machines for processing data, including by way of example, a programmable processor, a computer, a system on a chip, or multiple ones, or combinations of the foregoing The apparatus may include special purpose logic circuitry, e.g., an FPGA (field programmable gate array) and/or an ASIC (application specific integrated circuit). The apparatus may also include, in addition to hardware, code that creates an execution environment for the computer program in question, e.g., code that constitutes processor firmware, a protocol stack, a database management system, an operating system, a cross-platform runtime environment, a virtual machine, or any combination thereof. The apparatus and/or execution environment may realize various different computing model infrastructures, such as web services, distributed computing and grid computing infrastructures.

A computer program (i.e., a program, software, software application, script, code, or any combination thereof) may be written in any form of programming language, including compiled or interpreted languages, declarative or procedural languages, and it may be deployed in any form, including as a standalone program or as a module, component, subroutine, object, other unit suitable for use in a computing environment, or any combination thereof. A computer program may, but need not, correspond to a file in a file system. A program may be stored in a portion of a file that holds other programs or data (e.g., one or more scripts stored in a markup language document), in a single file dedicated to the program in question, and/or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes, operations, and/or logic flows described in this disclosure may be performed by one or more programmable processors executing one or more computer programs to perform actions by operating on input data and generating output. The processes, operations, and/or logic flows may also be performed by, and apparatus may also be implemented as, special purpose logic circuitry, e.g., an FPGA (field programmable gate array) or an ASIC (application specific integrated circuit).

Processors suitable for the execution of a computer program may include, by way of example, both general and special purpose microprocessors, and any one or more processors of any kind of digital computer. Generally, a processor can receive instructions and data from a read only memory and/or a random-access memory or both. The computer comprises a processor for performing actions in accordance with instructions and one or more memory devices for storing instructions and data. Generally, a computer may also include, or be operatively coupled to receive data from or transfer data to, or both, one or more mass storage devices for storing data, e.g., magnetic, magneto optical disks, optical disks, or any combination thereof. Moreover, a computer may be embedded in another device, e.g., a mobile telephone, a personal digital assistant (PDA), a mobile audio or video player, a game console, a Global Positioning System (GPS) receiver, a portable storage device (e.g., a universal serial bus (USB) flash drive), or any combination thereof. Devices suitable for storing computer program instructions and/or data may include all forms of non-volatile memory, media, and memory devices, including by way of example semiconductor memory devices, e.g., EPROM, EEPROM, and flash memory devices; magnetic disks, e.g., internal hard disks or removable disks; magneto optical disks; CD ROM and DVD-ROM disks; or any combination thereof. The processor and the memory may be supplemented by, or incorporated in, special purpose logic circuitry.

To provide for interaction with a user, embodiments of the subject matter described in this specification may be implemented on a computer having a display device, e.g., a CRT (cathode ray tube), LCD (liquid crystal display) monitor, OLED (organic light emitting diode) monitor, other form of display for displaying information to the user, or any combination thereof, and a keyboard and/or a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, tactile input, or any combination thereof. In addition, a computer may interact with a user by sending documents to and/or receiving documents from a device that is used by the user; for example, by sending web pages to a web browser on a user's client device in response to requests received from the web browser.

Having described certain embodiments of the methods and systems, it will now become apparent to one of skill in the art that other embodiments incorporating the concepts may be used. It should be understood that the systems described above may provide multiple ones of any or each of those components and these components may be provided on either a standalone machine or, in some embodiments, on multiple machines in a distributed system. The systems, devices, and/or methods, described elsewhere herein, may be implemented as a method, apparatus, and/or article of manufacture using programming and/or engineering techniques to produce software, firmware, hardware, or any combination thereof. In addition, the systems and methods, described elsewhere herein, may be provided as one or more computer-readable programs embodied on and/or in one or more articles of manufacture. The term “article of manufacture” as used herein is intended to encompass code and/or logic accessible from and embedded in one or more computer-readable devices, firmware, programmable logic, memory devices (e.g., EEPROMs, ROMs, PROMs, RAMS, SRAMs, or any combination thereof), hardware (e.g., integrated circuit chip, Field Programmable Gate Array (FPGA), Application Specific Integrated Circuit (ASIC), or any combination thereof), electronic devices, a computer readable non-volatile storage unit (e.g., CD-ROM, floppy disk, hard disk drive, or any combination thereof), or any combination thereof. The article of manufacture may be accessible from a file server providing access to the computer-readable programs via a network transmission line, wireless transmission media, signals propagating through space, radio waves, infrared signals, or any combination thereof. The article of manufacture may be a flash memory card and/or a magnetic tape. The article of manufacture may include hardware logic as well as software and/or programmable code embedded in a computer readable medium that is executed by a processor. In general, the computer-readable programs may be implemented in any programming language, such as LISP, PERL, C, C++, C#, PROLOG, in any byte code language such as JAVA, or any combination thereof. The software programs may be stored on or in one or more articles of manufacture as object code.

### Examples of Machine Learning Methodologies

As used in this disclosure and the appended claims, the terms “artificial intelligence,” “artificial intelligence techniques,” “artificial intelligence operation,” and “artificial intelligence algorithm” generally refer to any system and/or computational procedure that may take one or more actions that simulate human intelligence processes for enhancing or maximizing a chance of achieving a goal. The term “artificial intelligence” may include “generative modeling,” “machine learning” (ML), or “reinforcement learning” (RL).

As used in this disclosure and the appended claims, the terms “machine learning,” “machine learning techniques,” “machine learning operation,” and “machine learning model” generally refer to any system or analytical or statistical procedure that may progressively improve computer performance of a task. In some cases, ML may generally involve identifying and recognizing patterns in existing data in order to facilitate making predictions for subsequent data. ML may include a ML model (which may include, for example, a ML algorithm). Machine learning, whether analytical and/or statistical in nature, may provide deductive or abductive inference based on real or simulated data. The ML model may be a trained model. ML techniques may comprise one or more supervised, semi-supervised, self-supervised, or unsupervised ML techniques. For example, an ML model may be a trained model that is trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, ultra-deep learning, or any combination thereof. ML may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, generative adversarial networks, or any combination thereof.

Methods and/or systems of the disclosure can process and/or analyze one or more corneal images to determine an IOP of an eye for monitoring e.g., glaucoma, as described elsewhere herein. In some cases, the processing and/or analyzing of one or more corneal images may be conducted by way of one or more machine learning algorithms and/or one or more predictive models with instructions provided with one or more processors as described elsewhere herein. For example, one or more machine learning algorithms and/or predictive models may process one or more, or two or more features of the corneal images, described elsewhere herein.

In some cases, the subject's IOP may be determined and/or predicted with one or more machine learning algorithms and/or one or more predictive models with a sensitivity of at least about 70%, at least about 75%, at least about 80%, at least about 85% or at least about 90%.

In some cases, the IOP may be determined and/or predicted with one or more machine learning algorithms and/or one or more predictive models with a sensitivity of up to about 70%, up to about 75%, up to about 80%, up to about 85% or up to about 90%.

In some cases, the IOP may be determined and/or predicted with one or more machine learning algorithms and/or one or more predictive models with a specificity of at least about 70%, at least about 75%, at least about 80%, at least about 85% or at least about 90%.

In some cases, the IOP may be determined and/or predicted with one or more machine learning algorithms and/or one or more predictive models with a specificity of up to about 70%, up to about 75%, up to about 80%, up to about 85% or up to about 90%.

In some cases, the IOP may be determined and/or predicted with one or more machine learning algorithms and/or one or more predictive models with a positive predictive value of at least about 70%, at least about 75%, at least about 80%, at least about 85% or at least about 90%.

In some cases, the IOP may be determined and/or predicted with one or more machine learning algorithms and/or one or more predictive models with a positive predictive value of up to about 70%, up to about 75%, up to about 80%, up to about 85% or up to about 90%.

In some cases, the IOP may be determined and/or predicted with one or more machine learning algorithms and/or one or more predictive models with a negative predictive value of at least about 70%, at least about 75%, at least about 80%, at least about 85% or at least about 90%.

In some cases, the IOP may be determined and/or predicted with one or more machine learning algorithms and/or one or more predictive models with a negative predictive value of up to about 70%, up to about 75%, up to about 80%, up to about 85% or up to about 90%.

In some cases, the IOP may be determined and/or predicted with one or more machine learning algorithms and/or one or more predictive models with an Area Under the Receiver Operating Characteristic Curve (AUROC) of at least about 0.65, at least about 0.70, at least about 0.75, at least about 0.80, at least about 0.82, at least about 0.84, at least about 0.86, at least about 0.88, or at least about 0.90.

In some cases, the IOP may be determined and/or predicted with one or more machine learning algorithms and/or one or more predictive models with an Area Under the Receiver Operating Characteristic (AUROC) of up to about 0.65, up to about 0.70, up to about 0.75 up to about 0.80, up to about 0.82, up to about 0.84, up to about 0.86 up to about 0.88, or up to about 0.90.

An algorithm and/or predictive model can be implemented by way of software upon execution by one or more central processing unit(s). In some cases, the predictive model may comprise a machine learning predictive model. In some cases, the machine learning predictive model may comprise one or more statistical, machine learning, artificial intelligence algorithms, or any combination thereof. Examples of utilized algorithms, machine learning algorithms, and/or predictive models may include a support vector machine (SVM), a naïve Bayes classification, a random forest, a neural network (such as a deep neural network (DNN)), a recurrent neural network (RNN), a deep RNN, a long short-term memory (LSTM) recurrent neural network (RNN), decision tree algorithm, unsupervised clustering algorithm, a supervised clustering algorithm, unsupervised clustering algorithm, a regression algorithm, a gradient-boosting algorithm (e.g., a gradient-boosting implementation of a machine learning algorithm and/or predictive model such as a gradient-boosted decision trees), a gated recurrent unit (GRU), supervised learning algorithm, unsupervised learning algorithm, statistical, deep-learning algorithm for classification and/or regression, or any combination thereof. In some cases, the recurrent neural network may comprise units which can be LSTM units or GRU. In some cases, the predictive model and/or the machine learning algorithm may comprise an ensemble of one or more predictive models and/or machine learning algorithms

The machine learning predictive model may likewise involve the estimation of ensemble models, comprised of multiple machine learning algorithms and/or predictive models, and utilize techniques such as gradient boosting, for example in the construction of gradient-boosting decision trees. The machine learning predictive model may be trained using one or more training datasets corresponding to a model cornea. In some embodiments, the one or more training datasets may comprise distances in mm measured between implanted fluorescent particles and/or markers and corresponding IOPs of the eye.

Training records may be constructed from sequences of observations. Such sequences may comprise a fixed length for ease of data processing. For example, sequences may be zero-padded or selected as independent subsets of a single subject's records.

The one or more predictive models and/or one or more machine learning algorithms may process one or more input features to generate one or more output values comprising IOP of an eye. For example, such IOP may comprise a binary classification of a healthy/normal health state (e.g., absence of a disease or disorder) or an adverse health state (e.g., presence of a disease or disorder), a classification between a group of categorical labels (e.g., ‘no disease or disorder’, ‘apparent disease or disorder’, and ‘likely disease or disorder’), a likelihood (e.g., relative likelihood or probability) of developing a particular disease or disorder, a score indicative of a presence of disease or disorder, a score indicative of a level of systemic inflammation experienced by the patient, a ‘risk factor’ for the likelihood of mortality of the patient, a prediction of the time at which the patient is expected to have developed the disease or disorder, a confidence interval for any numeric predictions, or any combination thereof. Various predictive model and/or machine learning algorithms may be cascaded such that the output of one or more predictive models and/or one or more machine learning algorithms may be used as one or more input features to subsequent layers or subsections of the one or more predictive model and/or one or more machine learning algorithms.

In order to train the one or more predictive models and/or the one or more machine learning algorithms (e.g., by determining weights and correlations of the predictive model and/or the machine learning algorithm) to generate real-time classifications and/or predictions, the model can be trained using datasets (e.g., training datasets), described elsewhere herein. Such datasets may be sufficiently large to generate statistically significant classifications and/or predictions. For example, datasets may comprise databases of de-identified data including one or more distances between one or more particles and/or markers and associated IOP of eyes with the one or more particles and/or markers embedded.

Datasets, as described elsewhere herein, may be split into subsets (e.g., discrete or overlapping), such as a training dataset, a development dataset, and a test dataset. For example, a dataset may be split into a training dataset comprising 80% of the dataset, a development dataset comprising 10% of the dataset, and a test dataset comprising 10% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. Training sets (e.g., training datasets) may be selected by random sampling of a set of data corresponding to one or more subject cohorts to ensure independence of sampling. In some cases, training sets (e.g., training datasets) may be selected by proportionate sampling of a set of data corresponding to one or more subject cohorts to ensure independence of sampling.

To improve the accuracy of predictive model and/or machine learning algorithm predictions and reduce overfitting of the predictive model and/or machine learning algorithm, the datasets may be augmented to increase the number of samples within the training set. For example, data augmentation may comprise rearranging the order of observations in a training record. To accommodate datasets having missing observations, methods to impute missing data may be used, such as forward-filling, back-filling, linear interpolation, multi-task Gaussian processes, or any combination thereof. Datasets may be filtered to remove confounding factors. For example, within a database, a subset of subjects may be excluded.

Neural network techniques, such as dropout or regularization, may be used during training the one or more predictive models and/or one or more machine learning algorithms to prevent overfitting. The neural network may comprise a plurality of sub-networks, each of which is configured to generate a classification and/or prediction of a different type of output information (e.g., which may be combined to form an overall output of the neural network). The one or more predictive models and/or the one or more machine learning algorithms may alternatively utilize statistical or related algorithms including random forest, classification and regression trees, support vector machines, discriminant analyses, regression techniques, ensemble and gradient-boosted variations thereof, or any combination thereof.

When the one or more predictive models and/or the one or more machine learning algorithms generate a classification or a prediction of IOP, a notification (e.g., alert or alarm) may be generated and transmitted to a health care provider, such as a physician, nurse, health care personnel managing, or any combination thereof, treating a subject e.g., a subject within a hospital. Notifications may be transmitted via an automated phone call, a short message service (SMS), multimedia message service (MMS) message, an e-mail, an alert within a dashboard, or any combination thereof. The notification may comprise output information such as a prediction of IOP.

To validate the performance of the one or more predictive models and/or one more machine learning algorithms, different performance metrics may be generated. For example, an area under the receiver-operating curve (AUROC) may be used to determine the diagnostic and/or classification capability of the one or more predictive models and/or one or more machine learning algorithms. For example, the one or more predictive models and/or one or more machine learning algorithms may use classification thresholds which are adjustable, such that specificity and sensitivity are tunable, and the receiver-operating characteristic curve (ROC) can be used to identify the different operating points corresponding to different values of specificity and sensitivity of the one or more predictive models and/or one or more machine learning algorithms.

In some cases, such as when datasets are not sufficiently large, cross-validation may be performed to assess the robustness of one or more predictive models and/or one or more machine learning algorithms across different training and testing datasets.

To calculate performance metrics such as sensitivity, specificity, accuracy, positive predictive value (PPV), negative predictive value (NPV), AUPRC, AUROC, any combination thereof, or similar, the following definitions may be used. A “false positive” may refer to an outcome in which a positive outcome or result has been incorrectly or prematurely generated. A “true positive” may refer to an outcome in which positive outcome or result has been correctly generated. A “false negative” may refer to an outcome in which a negative outcome or result has been generated. A “true negative” may refer to an outcome in which a negative outcome or result has been generated.

The one or more predictive models and/or one or more machine learning algorithms may be trained until certain pre-determined conditions for accuracy and/or performance are satisfied, such as having minimum desired values corresponding to classification and/or diagnostic accuracy measures. Examples of diagnostic accuracy measures may include sensitivity, specificity, positive predictive value (PPV), negative predictive value (NPV), accuracy, area under the precision-recall curve (AUPRC), and area under the curve (AUC) of a Receiver Operating Characteristic (ROC) curve (AUROC) corresponding to the diagnostic accuracy of detecting or predicting IOP.

For example, such a pre-determined condition may be that the sensitivity of predicting the IOP comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the specificity of predicting the IOP comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the positive predictive value (PPV) of predicting the IOP comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the negative predictive value (NPV) of predicting the IOP comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the area under the curve (AUC) of a Receiver Operating Characteristic (ROC) curve (AUROC) of predicting the IOP comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a pre-determined condition may be that the area under the precision-recall curve (AUPRC) of predicting the IOP comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the IOP with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the IOP with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the IOP with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the IOP with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the IOP with an area under the curve (AUC) of a Receiver Operating Characteristic (ROC) curve (AUROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the IOP with an area under the precision-recall curve (AUPRC) of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

The training data sets may be collected from training subjects (e.g., humans). Each training subject has a diagnostic status indicating that they have been diagnosed and/or classified with a high IOP or have not been classified with a high IOP. The training procedure, as described elsewhere herein may be performed for each training subject in a plurality of training subjects.

In some embodiments, the machine learning analysis is performed by a device executing one or more programs (e.g., one or more programs stored in the Non-Persistent Memory or in the Persistent Memory) including instructions to perform the data analysis. In some embodiments, the data analysis is performed by a system comprising at least one processor (e.g., the processing core) and memory (e.g., one or more programs stored in the Non-Persistent Memory or in the Persistent Memory) comprising instructions to perform the data analysis.

Training the ML model may include, in some cases, selecting one or more untrained data models to train using a training data set. The selected untrained data models may include any type of untrained ML models for supervised, semi-supervised, self-supervised, unsupervised machine learning, or any combination thereof. The selected untrained data models may be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the ML model from the selected untrained data models may be selected, such as limits on the ML model complexity and/or limits on the ML model refinement past a certain point. The ML model may be trained (e.g., via a computer system such as a server) using the training data set. In some cases, a first subset of the training data set may be selected to train the ML model. The selected untrained data models may then be trained on the first subset of training data set using appropriate ML techniques, based upon the type of ML model selected and any conditions specified for training the ML model. In some cases, due to the processing power requirements of training the ML model, the selected untrained data models may be trained using additional computing resources (e.g., cloud computing resources). Such training may continue, in some cases, until at least one aspect of the ML model is validated and meets selection criteria to be used as a predictive model, described elsewhere herein.

In some cases, one or more aspects of the ML model may be validated using a second subset of the training data set (e.g., distinct from the first subset of the training data set) to determine accuracy and robustness of the ML model. Such validation may include applying the ML model to the second subset of the training data set to make predictions derived from the second subset of the training data. The ML model may then be evaluated to determine whether performance is sufficient based upon the derived predictions. The sufficiency criteria applied to the ML model may vary depending upon the size of the training data set available for training, the performance of previous iterations of trained models, user-specified performance requirements, or any combination thereof. If the ML model does not achieve sufficient performance, additional training may be performed. Additional training may include refinement of the ML model or retraining on a different first subset of the training dataset, after which the new ML model may again be validated and assessed. When the ML model has achieved sufficient performance, in some cases, the ML model may be stored for present and/or future use. The ML model may be stored as sets of parameter values or weights for analysis of further input (e.g., further relevant parameters to use as further predicted variables, further explanatory variables, further user interaction data, or any combination thereof), which may also include analysis logic or indications of model validity in some instances. In some cases, a plurality of ML models may be stored for generating predictions under different sets of input data conditions. In some embodiments, the ML model may be stored in a database (e.g., associated with a server).

While this disclosure contains many embodiment details, these should not be construed as limitations on the scope of any embodiments or of what may be claimed, but rather as descriptions of features specific to particular embodiments. Certain features described in this disclosure in the context of separate embodiments may also be implemented in combination in a single embodiment. Conversely, various features described in the context of a single embodiment may also be implemented in multiple embodiments separately or in any suitable sub combination. Moreover, although features may be described above as acting in certain combinations, one or more features from a claimed combination may in some cases be excised from the combination, and the claimed combination may be directed to a sub combination or variation of a sub combination.

Similarly, while operations are depicted in the drawings and/or disclosure in a particular order, this should not be understood as requiring that such operations be performed in the particular order shown and/or in sequential order, or that all illustrated operations be performed, to achieve desirable results. Certain operations or portions of a method may be repeated more than once. In certain circumstances, multitasking and/or parallel processing may be advantageous. Moreover, the separation of various system components in the embodiments described above should not be understood as requiring such separation in all embodiments, and it should be understood that the described program components and systems may generally be integrated in a single software product and/or packaged into multiple software products.

Thus, particular embodiments of the subject matter have been described. Other embodiments are within the scope of the following claims. In some cases, the actions recited in the claims may be performed in a different order and still achieve desirable results. In addition, the processes depicted in the accompanying figures do not necessarily require the particular order shown, or sequential order, to achieve desirable results. In certain embodiments, multitasking and parallel processing may be advantageous.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EMBODIMENTS

Numbered embodiment 1 comprises a method of determining an intraocular pressure (IOP) of an eye, where the method comprises: obtaining or receiving a first image of at least two markers embedded in the eye detected with a first detector and a second image of the at least two markers detected with a second detector, wherein an optical axis of the first detector is at an angle with respect to an optical axis of the second detector; and determining the IOP of the eye from a position of one or more of the at least two markers from the first image or the at least two markers from the second image.
Numbered embodiment 2 comprises the method of embodiment 1, wherein the at least two markers are embedded on a surface of the eye, in the cornea of the eye, in the lens of the eye, in a tissue adjacent to the eye, or any combination thereof.
Numbered embodiment 3 comprises the method of embodiment 1 or 2, wherein the at least two markers comprise fluorescent markers.
Numbered embodiment 4 comprises the method of any one of embodiments 1 to 3, further comprising providing a light source to the at least two markers embedded in the eye and obtaining or detecting the first image or the second image.
Numbered embodiment 5 comprises the method of embodiment 4, wherein the light source comprises a light emitting diode (LED).
Numbered embodiment 6 comprises the method of embodiment 4 or 5, wherein the light source, first detector, second detector, or a combination thereof, are contained at least partially within a chassis.
Numbered embodiment 7 comprises the method of embodiment 6, wherein the chassis comprises eyewear, eyeglasses, goggles, heads up display, virtual reality eyewear, augmented reality eyewear, or any combination thereof.
Numbered embodiment 8 comprises the method of any one of embodiments 1 to 7, wherein the first image of the at least two markers embedded in the eye is obtained or detected by the first detector through a first polarizing filter, and wherein the second image of the at least two markers embedded in the eye is obtained or detected by the second detector through a second polarizing filter.
Numbered embodiment 9 comprises the method of any one of embodiments 1 to 8, wherein the at least two markers comprise a particle.
Numbered embodiment 10 comprises the method of any one of embodiments 1 to 9, wherein the at least two markers comprise a first pair of markers and a second pair of markers.
Numbered embodiment 11 comprises the method of embodiment 10, wherein the IOP of the eye is determined from a ratio of a distance between a first marker and a second marker of the first pair of markers and a distance between a third marker and a fourth marker of the second pair of markers.
Numbered embodiment 12 comprises the method of any one of embodiments 1 to 11, wherein the position of the at least two markers is provided as an input to a machine learning algorithm or predictive model, wherein the machine learning algorithm or predictive model is trained to provide an output related to the TOP of the eye.
Numbered embodiment 13 comprises a system for determining an intraocular pressure (IOP) of an eye, where the system comprises: a detector optically coupled to an eye, wherein the detector is configured to detect a signal of at least two markers embedded in the eye; and one or more processors electrically coupled to the detector configured to process the signal of the two or more markers embedded in the eye and determine the IOP of the eye from a position of the two or more markers.
Numbered embodiment 14 comprises the system of embodiment 13, further comprising a light source optically coupled to the eye of the subject.
Numbered embodiment 15 comprises the system of embodiment 14, wherein the light source comprises a light emitting diode (LED).
Numbered embodiment 16 comprises the system of embodiment 14 or 15, wherein the light source and detector are contained at least partially in a chassis, wherein the chassis is positioned at a distance up to about 2 centimeters from a tangential surface of the eye.
Numbered embodiment 17 comprises the system of embodiment 16, wherein the chassis comprises eyewear, eyeglasses, googles, heads up display, virtual reality eyewear, augmented reality eyewear, or any combination thereof.
Numbered embodiment 18 comprises the system of any one of embodiments 13 to 17, wherein the detector comprises a first detector and a second detector.
Numbered embodiment 19 comprises the system of embodiment 18, wherein an optical axis of the first detector is at an angle with respect to an optical axis of the second detector.
Numbered embodiment 20 comprises the system of any one of embodiments 13 to 19, wherein the at least two markers are positioned at a distance of up to about 6 millimeters from each other.
Numbered embodiment 21 comprises the system of any one of embodiments 13 to 20, wherein the detector comprises a camera, a charge coupled device (CCD) sensor, complementary metal oxide semiconductor (CMOS) sensor, or any combination thereof.
Numbered embodiment 22 comprises a system for determining an intraocular pressure (IOP) of an eye, where the system comprises: one or more processors and a memory storing one or more programs for execution by the one or more processors, the one or more programs comprising instructions to: (i) obtain or receive one or more images of at least two markers embedded in the eye, wherein the one or more images are detected with a first detector and a second detector, and wherein an optical axis of the first detector is at an angle with respect to an optical axis of the second detector; and (ii) determine the TOP of the eye from a distance between the at least two markers in the one or more images.
Numbered embodiment 23 comprises the system of embodiment 22, wherein the one or more images are obtained or received from a server, cloud base storage, eyewear device, or any combination thereof.
Numbered embodiment 24 comprises the system of embodiment 23, wherein the eyewear device comprises virtual reality eyewear, augmented reality eyewear, or a combination thereof.
Numbered embodiment 25 comprises the system of embodiment 23 or 24, wherein the one or more processors are on the eyewear device, a remote processing device, a remote server, a cloud server, or any combination thereof.
Numbered embodiment 26 comprises the system of any one of embodiments 22 to 25, wherein the at least two markers are positioned at a distance of up to about 6 millimeters from each other.
Numbered embodiment 27 comprises a method of training a machine learning algorithm or predictive model to determine an intraocular pressure (IOP) of an eye, where the method comprises: receiving or obtaining one or more images of at least two markers embedded in one or more eyes and a corresponding IOP of the one or more eyes; and training an untrained or partially untrained machine learning algorithm or predictive model with the one or more images of the at least two markers embedded in the one or more eyes and the corresponding IOP of the one or more eyes thereby producing a trained machine learning algorithm or trained predictive model.
Numbered embodiment 28 comprises the method of embodiment 27, wherein the untrained or partially untrained machine learning algorithm or predictive model is further trained on a distance between the at least two markers in the one or more images of the at least two markers embedded in the one or more eyes.
Numbered embodiment 29 comprises the method of embodiment 27 or 28, wherein the one or more images of the at least two markers embedded in the one or more eyes is detected with a first detector and a second detector, and wherein an optical axis of the first detector is at an angle with respect to an optical axis of the second detector.
Numbered embodiment 30 comprises the method of any one of embodiments 27 to 29, wherein the at least two markers are positioned at a distance of up to about 6 millimeters from each other.
Numbered embodiment 31 comprises the method of any one of embodiments 27 to 30, wherein the at least two markers are on a surface of the eye, in the cornea of the eye, in the lens of the eye, in a tissue adjacent to the eye, or any combination thereof.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

References to “or” may be construed as inclusive so that any terms described using “or” may indicate any of a single, more than one, and all of the described terms.

As used in the specification and claims, the singular forms “a”, “an” and “the” include plural references unless the context clearly dictates otherwise. For example, the term “a sample” includes a plurality of samples, including mixtures thereof.

“Microparticles,” “fluorescent particles,” “beads,” fluorescent beads,” “particles,” and “markers” are used interchangeably throughout the application.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and/or protozoa. The subject can be tissues, cells and/or their progeny of a biological entity obtained in vivo or cultured in vitro. The subject can be a mammal. The mammal can be a human. The subject may be diagnosed or suspected of being at high risk for a disease e.g., glaucoma. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated e.g., lowering IOP for one or more subjects. Also, a therapeutic benefit can be achieved with the eradication or amelioration of one or more of the physiological symptoms associated with the underlying disorder such that an improvement is observed in one or more subjects' IOP, notwithstanding that the subject may still be afflicted with the underlying disorder. A prophylactic effect includes delaying, preventing, and/or eliminating the appearance of a disease and/or condition, delaying and/or eliminating the onset of symptoms of a disease or condition, slowing, halting, and/or reversing the progression of a disease and/or condition, or any combination thereof. For prophylactic benefit, a subject at risk of developing a particular disease, and/or to a subject reporting one or more of the physiological symptoms of a disease may undergo treatment, even though a diagnosis of this disease may not have been made.

1.-31. (canceled)

32. A method of determining an intraocular pressure (IOP) of an eye, the method comprising:
a. obtaining or receiving a first image of at least two markers embedded in the eye detected with a first detector and a second image of the at least two markers detected with a second detector, wherein an optical axis of the first detector is at an angle with respect to an optical axis of the second detector; and
b. determining the TOP of the eye from a position of one or more of the at least two markers from the first image or the at least two markers from the second image.

33. The method of claim 32, wherein the at least two markers are embedded on a surface of the eye, in the cornea of the eye, in the lens of the eye, in a tissue adjacent to the eye, or any combination thereof.

34. The method of claim 32, wherein the at least two markers comprise fluorescent markers.

35. The method of claim 32, further comprising providing a light source to the at least two markers embedded in the eye and obtaining or detecting the first image or the second image.

36. The method of claim 32, wherein the first image of the at least two markers embedded in the eye is obtained or detected by the first detector through a first polarizing filter, and wherein the second image of the at least two markers embedded in the eye is obtained or detected by the second detector through a second polarizing filter.

37. The method of claim 32, wherein the at least two markers comprise a particle.

38. The method of claim 32, wherein the at least two markers comprise a first pair of markers and a second pair of markers.

39. The method of claim 38, wherein the TOP of the eye is determined from a ratio of a distance between a first marker and a second marker of the first pair of markers and a distance between a third marker and a fourth marker of the second pair of markers.

40. The method of claim 32, wherein the position of the at least two markers is provided as an input to a machine learning algorithm or predictive model, wherein the machine learning algorithm or predictive model is trained to provide an output related to the TOP of the eye.

41. The method of claim 40, wherein the machine learning algorithm or predictive model is further trained on a distance between the at least two markers in one or more images of the at least two markers embedded in the eye.

42. A system for determining an intraocular pressure (IOP) of an eye, the system comprising:
a. a detector optically coupled to an eye, wherein the detector is configured to detect a signal of at least two markers embedded in the eye; and
b. one or more processors electrically coupled to the detector configured to process the signal of the two or more markers embedded in the eye and determine the TOP of the eye from a position of the two or more markers.

43. The system of claim 42, further comprising a light source optically coupled to the eye.

44. The system of claim 43, wherein the light source comprises a light emitting diode (LED).

45. The system of claim 43, wherein the light source and the detector are contained at least partially in a chassis, wherein the chassis is positioned at a distance up to about 2 centimeters from a tangential surface of the eye.

46. The system of claim 45, wherein the chassis comprises eyewear, eyeglasses, googles, a heads up display, virtual reality eyewear, augmented reality eyewear, or any combination thereof.

47. The system of claim 46, wherein the chassis is electronically coupled to one or more processors on the chassis, a remote processing device, a remote server, a cloud server, or any combination thereof.

48. The system of claim 42, wherein the at least two markers are positioned at a distance of up to about 6 millimeters from each other.

49. The system of claim 42, wherein the detector comprises a camera, a charge coupled device (CCD) sensor, a complementary metal oxide semiconductor (CMOS) sensor, or any combination thereof.

50. The system of claim 42, wherein the at least two markers are on a surface of the eye, in the cornea of the eye, in the lens of the eye, in a tissue adjacent to the eye, or any combination thereof.
